(19) United States
(12) Patent Application Publication
(10) Pub. No.: US 2008/0119456 A1
(43) Pub. Date: May 22, 2008

(54) Substituted Thiazoleacetic Acid as Crth2 Ligands

(76) Inventors: Trond Ulven, Hoersholm (DK); Thomas Frimurer, Hoersholm (DK); Oystein Rist, Hoersholm (DK); Evi Kostenis, Hoersholm (DK); Thomas Hogberg, Hoersholm (DK); Jean-Marie Receveur, Hoersholm (DK); Marie Grimstrup, Hoersholm (DK)

Correspondence Address: BANNER & WITCOFF, LTD., 1100 13th STREET, N.W., SUITE 1200, WASHINGTON, DC, 20005-4051, US

(21) Appl. No.: 11/597,839

(22) PCT Filed: May 30, 2005
(86) PCT No.: PCT/EP05/05882

§ 371 Date: Sep. 14, 2007

(30) Foreign Application Priority Data
May 29, 2004 (GB) 0412198.4
Jun. 24, 2004 (GB) 0414194.1
Oct. 29, 2004 (GB) 0424016.4

(51) Int. Cl. A61K 31/53; A61K 31/506; A61K 31/55; C07D 417/02; C07D 413/02; C07D 403/02

(52) U.S. Cl. 514/217.04; 514/242; 514/256; 514/340; 514/365; 514/374; 514/381; 514/397; 544/183; 544/333; 546/284.4; 548/181; 548/215; 548/253; 514/217.06; 514/217.08; 540/597; 540/598

(57) ABSTRACT

Compounds of formula (I) are useful for the treatment of disease responsive to modulation of CRTH2 receptor activity, such as asthma, rhinitis, allergic airway syndrome, and allergic rhinobronchitis; wherein X₁ is —S—, —O—, —N═N—. —NR₇—, —CR₇═CR₈—, —CR₇═N—, wherein R₇ and R₈ are independently hydrogen or C₁-C₃ alkyl; A is a carboxyl group —COOH, or a carboxyl bioisostere; rings Ar² and Ar³ each independently represent a phenyl or 5- or 6-membered monocyclic heteroaryl ring, or a bicyclic ring system consisting of a 5- or 6-membered carbocyclic or heterocyclic ring which is benz-fused or fused to a 5- or 6-membered monocyclic heteroaryl ring, said ring or ring system being optionally substituted; ring B is as defined for Ar² and Ar³, or an optionally substituted N-pyrrolidinyl, N-piperidinyl or N-azepinyl ring; s is 0 or 1; L1, L2 and L4 are linker radicals as defined in the description; Q₁ and Q₂ represent substituents as defined in the description.

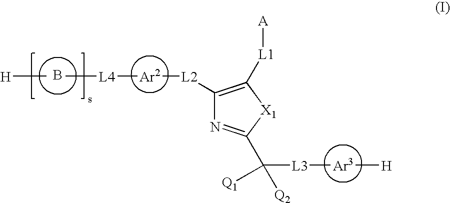

This invention relates to a class of compounds which are ligands of the CRTH2 receptor (Chemoattractant Receptor-homologous molecule expressed on T Helper cells type 2), and their use in the treatment of diseases responsive to modulation of CRTH2 receptor activity, principally diseases having a significant inflammatory component. The invention also relates to novel members of that class of ligands and pharmaceutical compositions containing them.

### BACKGROUND TO THE INVENTION

The natural ligand of the G-protein coupled receptor CRTH2 is prostaglandin D2. As its name implies, CRTH2 is expressed on T helper cells type 2 (TH2 cells) but it is also known to be expressed on eosinophils and basophil cells. Cell activation as a result of binding of PGD2 to the CRTH2 receptor results in a complex biological response, including release of inflammatory mediators. Elevated levels of PGD2 are therefore associated with many diseases which have a strong inflammatory component, such as asthma, rhinitis and allergies. Blocking binding of PGD2 to the CRTH2 receptor is therefore a useful therapeutic strategy for treatment of such diseases.

Some small molecule ligands of CRTH2, apparently acting as antagonists of PGD2, are known, for example as proposed in the following patent publications: WO 03/097042, WO 03/097598, WO 03/066046, WO 03/066047, WO 03/101961, WO 03/101981, GB 2388540, WO 04/089885 and WO 05/018529.

NSAIDs (non-steroidal anti-inflammatory drugs) constitute another class of anti-inflammatory agents. One NSAID is 4-(4-chlorophenyl)-2-phenyl thiazole-5 acetic acid (fentiazac). Some other thiazole compounds have been investigate as anti-inflammatory agents (see for example Nagatomi et. al. Arzneimittel-Forschung (1984), 34(5), 599-603; Bonina et. al. Farmaco, Edizione Scientifica (1987), 42(12, 905-13; Gieldanowski et. al. Archivum Immunologiae et Therapiae Experimentalis, 1978, 26, 921-929; Brown et. al. J. Med. Chem., 1974, Vol 17, No. 11 1177-1181; Attimarad et. al. Asian J. Chem. 2004, 16(1), 179-182; Japanese Patent publications JP07149745 and 07149746; and International patent publications WO 9727190, WO 2003103657

### BRIEF DESCRIPTION OF THE INVENTION

The structures of the PGD2 antagonist compounds referred to in the foregoing publications have a bicyclic or tricyclic core ring system related to the indole core of indomethacin, a known anti-inflammatory agent, now known to bind to CRTH2. The present invention arises from the identification of a class of compounds having a 5- or 6-membered nitrogen-containing monocyclic core such as a thiazole ring, whose substituent moieties are orientated by the monocyclic core, to interact with and bind to CRTH2. The class of compounds with which this invention is concerned are thus capable of modulating CRTH2 activity, and are useful in the treatment of diseases which benefit from such modulation, for example asthma, allergy and rhinitis.

### DETAILED DESCRIPTION OF THE INVENTION

According to the present invention, there is provided a compound of formula (I) or a salt, hydrate or solvate thereof:

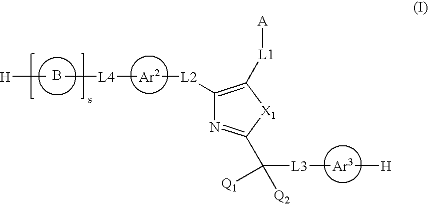

wherein

### X₁ is —S—, —O—, —N═N—. —NR₇—, —CR₇═CR₈—, —CR₇═N—, wherein R₇ and R₈ are independently hydrogen or C₁-C₃ alkyl;

### A is a carboxyl group —COOH, or a carboxyl bioisostere;

rings Ar² and Ar³ each independently represent a phenyl or 5- or 6-membered monocyclic heteroaryl ring, or a bicyclic ring system consisting of a 5- or 6-membered carbocyclic or heterocyclic ring which is benz-fused or fused to a 5- or 6-membered monocyclic heteroaryl ring, said ring or ring system being optionally substituted;
ring B is as defined for Ar² and Ar³, or an optionally substituted N-pyrrolidinyl, N-piperidinyl or N-azepinyl ring;
s is 0 or 1;

### L1 represents a divalent radical of formula -(Alk¹)ₘ- and L2 and L4 each independently represents a divalent radical of formula -(Alk¹)ₘ-(Z)ₙ-(Alk²)ₚ- wherein

m, n and p are independently 0 or 1,
Alk¹ and Alk² are independently optionally substituted straight or branched chain C₁-C₃ alkylene or C₂-C₃ alkenylene radicals which may contain a compatible —O—, —S— or —NR— link wherein R is hydrogen or C₁-C₃ alkyl, and
Z is —O—; —S—; —C(═O)—; —SO₂—; —SO—; —NR—, —NRSO₂—, —C(═O)NR—, —NRCONH—, NRC(═NR)NH—, or ═N—NR— wherein R is hydrogen or C₁-C₃alkyl; or a divalent 5- or 6-membered monocyclic carbocyclic or heterocyclic radical;
   L3 represents a divalent radical of formula -(Alk³)ₘ-(Z)ₙ-(Alk²)ₚ- wherein m, n, p, Alk² and Z are as defined in relation to L2 and L4, and Alk3 is an optionally substituted straight or branched chain C₁-C₂ alkylene or C₁-C₂ alkenylene radical which may contain a compatible —O—, —S— or —NR— link wherein R is hydrogen or C₁-C₃alkyl;

### Q₁ represents hydrogen or (C₁-C₆)alkyl;

### Q₂ represents

(i) (C₁-C₆)alkyl, (C₁-C₆)alkoxy, hydroxy, hydroxy(C₁-C₆)alkyl, nitrile (—CN), phenyl, phenoxy, monocyclic heteroaryl or heteroaryloxy with 5 or 6 ring atoms, —CONR^{A}R^{B}, —NR^{B}COR^{A}, —NR^{B}SO₂R^{A} or —NR^{A}CONR^{A}R^{B} wherein R^{A} and R^{B} are independently hydrogen or a (C₁-C₆)alkyl group, or R^{A} and R^{B} are linked to the same N atom to form a cyclic amino ring, and when Q is phenyl, phenoxy or monocyclic heteroaryl or heteroaryloxy with 5 or 6 ring atoms the phenyl or heteroaryl ring is optionally substituted by any of (C₁-C₆)alkyl, (C₁-C₆)alkoxy, hydroxy, hydroxy(C₁-C₆)alkyl, (C₁-C₃)alkylthio, halo, fully or partially fluorinated (C₁-C₃)alkyl, (C₁-C₃)alkoxy or (C₁-C₃)alkylthio, trifluoromethylthio, nitro, nitrile (—CN), —COOR^{A}, —COR^{A}, —OCOR^{A}, —SO₂R^{A}, —CONR^{A}R^{B}, —SO₂NR^{A}R^{B}, —NR^{A}R^{B}, —NR^{B}COR^{A}, —NR^{B}COOR^{A}, —NR^{B}SO₂R^{A} or —NR^{A}CONR^{A}R^{B} wherein R^{A} and R^{B} are independently hydrogen or a (C₁-C₆)alkyl group, or R^{A} and R^{B} are linked to the same N atom to form a cyclic amino ring, or
(ii) hydrogen, but only when, in L3, Z represents an optionally substituted divalent 5- or 6-membered monocyclic carbocyclic or heterocyclic radical;
or Q₁ and Q₂ taken together with the carbon atom to which they are attached form a C₃-C₆ cycloalkyl ring or a monocyclic non-aromatic heterocyclic ring with 4-6 ring atoms;
and wherein the total length of L2 and L3 does not exceed that of an unbranched saturated chain of 10 carbon atoms

In a narrower definition of the compounds (I), (i) the length of each of L2, L3 and L4 does not exceed that of an unbranched saturated chain of 5 atoms and (ii) the total length of L2, L3 and L4 does not exceed that of an unbranched saturated chain of 7 atoms, and (iii) none of L1, L2, L3 and L4 includes more than two R substituents different from hydrogen.

The compounds with which the invention is concerned are defined by reference to formula (I) as a result of studies towards elucidation of the ligand binding site of CRTH2. Such studies led to the overall conclusion that a general pharmacophore comprising one negatively charged moiety, represented by AL1-, and two aromatic and/or hydrophobic moieties, represented by H(B)ₛL4Ar²L2 and either the ring containing X₁ or the ring containing X₁ together with the H(Ar³)L3C(Q₁)(Q₂)- fragment, oriented in an approximate triangle, would form an arrangement for interaction with the receptor binding site. It was concluded that the substituent groupings AL1-, H(B)ₛL4Ar²L2- should be on adjacent ring atoms of the ring containing X₁. The linkers L1, L2, L3 and L4 provide some flexibility to the molecule to facilitate optimum binding. The restrictions on the lengths of, and substitutions in, the linkers L2, L3 and L4 are in order to restrict the total molecular size and complexity of structures for use in accordance with the invention. For the avoidance of doubt, the total length of L2 and L3 is, for the purposes of this description and claims, the sum n2+n3, where n2 is the number of connected atoms in the shortest chain of atoms from terminal atom to terminal atom of linker L2, and n3 is the number of connected atoms in the shortest chain of atoms from terminal atom to terminal atom of linker L2. Preferably the compounds with which the invention is concerned should have a molecular weight of no more than 600. Optional substituents in any element of the compounds (I) are permitted as in the definition of compounds (I). Such substituents can modulate pharmacokinetic and solubility properties, as well as picking up additional binding interactions with the receptor.

In another aspect, the invention provides the use of a compound as defined and discussed herein in the manufacture of a composition for the treatment of disease responsive to modulation of CRTH2 receptor activity.

In another aspect, the invention provides a method of treatment of a subject suffering from a disease responsive to modulation of CRTH2 receptor activity, which comprised administering to the subject an amount of a compound (I) as defined and discussed herein, effective to ameliorate the disease.

In particular, compounds with which the invention is concerned are useful in the treatment of disease associated with elevated levels of prostaglandin D2 (PGD2) or one or more active metabolites thereof.

Examples of such diseases include asthma, rhinitis, allergic airway syndrome, allergic rhinobronchitis, bronchitis, chronic obstructive pulmonary disease (COPD), nasal polyposis, sarcoidosis, farmer's lung, fibroid lung, cystic fibrosis, chronic cough, conjunctivitis, atopic dermatitis, Alzheimer's disease, amyotrophic lateral sclerosis, AIDS dementia complex, Huntington's disease, frontotemporal dementia, Lewy body dementia, vascular dementia, Guillain-Barre syndrome, chronic demyelinating polyradiculoneurophathy, multifocal motor neuropathy, plexopathy, multiple sclerosis, encephalomyelitis, panencephalitis, cerebellar degeneration and encephalomyelitis, CNS trauma, migraine, stroke, rheumatoid arthritis, ankylosing spondylitis, Behget's Disease, bursitis, carpal tunnel syndrome, inflammatory bowel disease, Crohn's disease, ulcerative colitis, dermatomyositis, Ehlers-Danlos Syndrome (EDS), fibromyalgia, myofascial pain, osteoarthritis (OA), osteonecrosis, psoriatic arthritis, Reiter's syndrome (reactive arthritis), sarcoidosis, scleroderma, Sjogren's Syndrome, soft tissue disease, Still's Disease, tendinitis, polyarteritis Nodossa, Wegener's Granulomatosis, myositis (polymyositis dermatomyositis), gout, atherosclerosis, lupus erythematosus, systemic lupus erythematosus (SLE), type I diabetes, nephritic syndrome, glomerulonephritis, acute and chronic renal failure, eosinophilia fascitis, hyper IgE syndrome, sepsis, septic shock, ischemic reperfusion injury in the heart, allograft rejection after transplantations, and graft versus host disease.

However, the compounds with which the invention is concerned are primarily of value for the treatment asthma, rhinitis, allergic airway syndrome, and allergic rhinobronchitis

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein the term “divalent (Cₐ-C_{b})alkylene radical” wherein a and b are integers refers to a saturated hydrocarbon chain having from a to b carbon atoms and two unsatisfied valences.

As used herein the term “(Cₐ-C_{b})alkenyl” wherein a and b are integers refers to a straight or branched chain alkenyl moiety having from a to b carbon atoms having at least one double bond of either E or Z stereochemistry where applicable. The term includes, for example, vinyl, allyl, 1- and 2-butenyl and 2-methyl-2-propenyl.

As used herein the term “divalent (Cₐ-C_{b})alkenylene radical” means a hydrocarbon chain having from a to a carbon atoms, at least one double bond, and two unsatisfied valences.

As used herein the term “Cₐ-C_{b} alkynyl” wherein a and b are integers refers to straight chain or branched chain hydrocarbon groups having from two to six carbon atoms and having in addition one triple bond. This term would include for example, ethynyl, 1- and 2-propynyl, 1-, 2- and 3-butynyl, 1,2-, 3- and 4-pentynyl, 1-, 2-, 3-, 4- and 5-hexynyl, 3-methyl-1-butynyl, 1-methyl-2-pentynyl.

As used herein the term “divalent (Cₐ-C_{b})alkynylene radical” wherein a and b are integers refers to a divalent hydrocarbon chain having from 2 to 6 carbon atoms, at least one triple bond, and two unsatisfied valences.

As used herein the term “carbocyclic” refers to a mono-, bi- or tricyclic radical having up to 16 ring atoms, all of which are carbon, and includes aryl and cycloalkyl.

As used herein the term “cycloalkyl” refers to a monocyclic saturated carbocyclic radical having from 3-8 carbon atoms and includes, for example, cyclopropyl, cyclobutyl, cyclopentyl, cyclohexyl, cycloheptyl and cyclooctyl.

As used herein the unqualified term “aryl” refers to a mono-, bi- or tri-cyclic carbocyclic aromatic radical, and includes radicals having two monocyclic carbocyclic aromatic rings which are directly linked by a covalent bond. Illustrative of such radicals are phenyl, biphenyl and napthyl.

As used herein the unqualified term “heteroaryl” refers to a mono-, bi- or tri-cyclic aromatic radical containing one or more heteroatoms selected from S, N and O, and includes radicals having two such monocyclic rings, or one such monocyclic ring and one monocyclic aryl ring, which are directly linked by a covalent bond. Illustrative of such radicals are thienyl, benzothienyl, furyl, benzofuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyridazinyl, triazinyl, indolyl and indazolyl.

As used herein the unqualified term “heterocyclyl” or “heterocyclic” includes “heteroaryl” as defined above, and in addition means a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzofuranyl, pyranyl, isoxazolyl, benzimidazolyl, methylenedioxyphenyl, ethylenedioxyphenyl, maleimido and succinimido groups.

The term “carboxyl bioisostere” is a term familiar to medicinal chemists (see for example “The Organic Chemistry of Drug Design and Drug Action”, by Richard B. Silverman, pub. Academic Press, 1992), and refers to a group which has similar acid-base characteristics to those of a carboxyl group. Well known carboxyl bioisosteres include —SO₂NHR or —P(═O)(OH)(OR) wherein R is, for example, hydrogen methyl or ethyl, —SO₂OH, —P(═O)(OH)(NH₂), —C(═O)NHCN and groups of formulae:

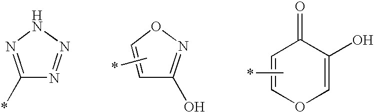

Unless otherwise specified in the context in which it occurs, the term “substituted” as applied to any moiety herein means substituted with up to four compatible substituents, each of which independently may be, for example, (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, halo (including fluoro, bromo and chloro), fully or partially fluorinated (C₁-C₃)alkyl, (C₁-C₃)alkoxy or (C₁-C₃)alkylthio such as trifluoromethyl, trifluoromethoxy, and trifluoromethylthio, nitro, nitrile (—CN), oxo, phenyl, phenoxy, monocyclic heteroaryl or heteroaryloxy with 5 or 6 ring atoms, —COOR^{A}, —COR^{A}, —OCOR^{A}, —SO₂R^{A}, —CONR^{A}R^{B}, —SO₂NR^{A}R^{B}, —NR^{A}R^{B}, OCONR^{A}R^{B}, —NR^{B}COR^{A}, —NR^{B}COOR^{A}, —NR^{B}SO₂OR^{A} or —NR^{A}CONR^{A}R^{B} wherein R^{A} and R^{B} are independently hydrogen or a (C₁-C₆)alkyl group or, in the case where R^{A} and R^{B} are linked to the same N atom, R^{A} and R^{B} taken together with that nitrogen may form a cyclic amino ring. Where the substituent is phenyl, phenoxy or monocyclic heteroaryl or heteroaryloxy with 5 or 6 ring atoms, the phenyl or heteroaryl ring thereof may itself be substituted by any of the above substituents except phenyl phenoxy, heteroaryl or heteroaryloxy. An “optional substituent” may be one of the foregoing substituent groups.

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Those compounds (I) which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic, p-toluenesulphonic, benzoic, benzenesulfonic, glutamic, lactic, and mandelic acids and the like.

Compounds with which the invention is concerned which may exist in one or more stereoisomeric form, because of the presence of asymmetric atoms or rotational restrictions, can exist as a number of stereoisomers with R or S stereochemistry at each chiral centre or as atropisomeres with R or S stereochemistry at each chiral axis. The invention includes all such enantiomers and diastereoisomers and mixtures thereof.

Use of prodrugs, such as esters, of compounds (I) with which the invention is concerned is also part of the invention.

For use in accordance with the invention, the following structural characteristics are currently preferred, in any compatible combination, in the compounds (I):
Q₁ is hydrogen, and Q₂ is phenyl or monocyclic heteroaryl with 5 or 6 ring atoms optionally substituted by any of (C₁-C₆)alkyl, (C₁-C₆)alkoxy, hydroxy, hydroxy(C₁-C₆)alkyl, (C₁-C₃)alkylthio, halo, fully or partially fluorinated (C₁-C₃)alkyl, (C₁-C₃)alkoxy or (C₁-C₃)alkylthio, trifluoromethylthio, nitro, nitrile (—CN), —COOR^{A}, —COR^{A}, —OCOR^{A}, —SO₂R^{A}, —CONR^{A}R^{B}, —SO₂NR^{A}R^{B}, NR^{A}R^{b}, —NR^{B}COR^{A}, —NR^{B}COOR^{A}, —NR^{B}SO₂OR^{A} or —NR^{A}CONR^{A}R^{B} wherein R^{A} and R^{B} are independently hydrogen or a (C₁-C₆)alkyl group, or R^{A} and R^{B} are linked to the same N atom to form a cyclic amino ring; or
Q₁ is hydrogen, and Q₂ is phenyl, optionally substituted by any of fluoro, chloro, bromo, (C₁-C₃)alkyl, trifluoromethyl, (C₁-C₃)alkoxy, trifluoromethoxy, trifluoromethylthio, dimethylamino, cyano, (C₁-C₃alkyl)SO₂—, NH₂SO₂—, (C₁-C₃alkyl)NHSO₂—, (C₁-C₃alkyl)₂NSO₂—, —CONR^{A}R^{B}, and —NR^{B}COR^{A}. wherein R^{A} and R^{B} are independently hydrogen or a (C₁-C₆)alkyl group, or R^{A} and R^{B} are linked to the same N atom to form a cyclic amino ring.
L3 is —CH₂—, —O—, —S—, —SO₂—, —NHC(═O)—, —CH═CH—, —NR₁₁—, or —NR₁₁CH₂—, wherein R₁₁ is hydrogen or C₁-C₃ alkyl; or
L3 represents a divalent radical of formula -(Alk³)ₘ-(Z)ₙ-(Alk²)ₚ- wherein m is 0, n is 1, and Z is a phenylene radical optionally substituted by one or more of fluoro, chloro, bromo, (C₁-C₃)alkyl, trifluoromethyl, (C₁-C₃)alkoxy, trifluoromethoxy, trifluoromethylthio, dimethylamino, cyano, (C₁-C₃alkyl)SO₂—, NH₂SO₂—, (C₁-C₃alkyl)NHSO₂—, (C₁-C₃alkyl)₂NSO₂—, —CONR^{A}R^{B}, and —NR^{B}COR^{A}. wherein R^{A} and R^{B} are independently hydrogen or a (C₁-C₆)alkyl group, or R^{A} and R^{B} are linked to the same N atom to form a cyclic amino ring. In these cases, Z may be, for example, a 1,2-phenylene radical optionally substituted by one or more of fluoro, chloro, bromo, (C₁-C₃)alkyl, trifluoromethyl, (C₁-C₃)alkoxy, trifluoromethoxy, trifluoromethylthio, dimethylamino, cyano, (C₁-C₃alkyl)SO₂—, NH₂SO₂—, (C₁-C₃alkyl)NHSO₂—, (C₁-C₃alkyl)₂NSO₂—, —CONR^{A}R^{B}, and —NR^{B}COR^{A} wherein R^{A} and R^{B} are independently hydrogen or a (C₁-C₆)alkyl group, or R^{A} and R^{B} are linked to the same N atom to form a cyclic amino ring.
In particular, Q₁ may be hydrogen, X₁ may be —S—, and A is may be carboxyl group —COOH;
L1 is a bond, —CR₁₁R₁₂—, *—CH₂CR₁₁R₁₂—, *—OCR₁₁R₁₂—, *—SCR₁₁R₁₂—, *—NR₁₁CH₂— or —NR₁₁— wherein R₁₁ and R₁₂ are independently hydrogen or C₁-C₃ alkyl, the bond marked with an asterisk being the one connected to the ring containing X¹. For example L1 may be —CH₂— or —CH(CH₃)—.
Ar³ is phenyl, thienyl, naphthyl or 2-, 3- or 4-pyridyl, any of which is optionally substituted, for example by one or more substituents selected from fluoro, chloro, bromo, (C₁-C₃)alkyl, trifluoromethyl, (C₁-C₃)alkoxy, trifluoromethoxy, trifluoromethylthio, dimethylamino, cyano, (C₁-C₃alkyl)SO₂—, NH₂SO₂—, (C₁-C₃alkyl)NHSO₂—, (C₁-C₃alkyl)₂NSO₂—, —CONR^{A}R^{B}, and —NR^{B}COR^{A} wherein R^{A} and R^{B} are independently hydrogen or a (C₁-C₆)alkyl group, or R^{A} and R^{B} are linked to the same N atom to form a cyclic amino ring;
L2 is a bond and Ar² is an optionally substituted phenyl, thienyl, furanyl, pyrrolyl or pyridyl ring, optional substituents being selected from fluoro, chloro, bromo, (C₁-C₃)alkyl, trifluoromethyl, (C₁-C₃)alkoxy, trifluoromethoxy, trifluoromethylthio, dimethylamino, cyano, (C₁-C₃alkyl)SO₂—, NH₂SO₂—, (C₁-C₃alkyl)NHSO₂—, (C₁-C₃alkyl)₂NSO₂—, —CONR^{A}R^{B}, and —NR^{B}COR^{A} wherein R^{A} and R^{B} are independently hydrogen or a (C₁-C₆)alkyl group, or R^{A} and R^{B} are linked to the same N atom to form a cyclic amino ring;
s is 0.

One preferred subclass of the compounds with which the invention is concerned consists of compounds of formula (IA), and salts, hydrates and solvates thereof:

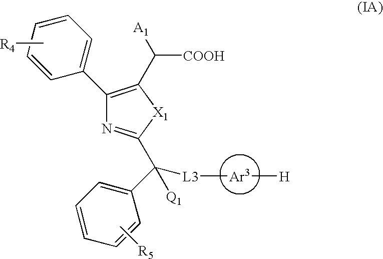

wherein A₁ is hydrogen or methyl, X₁, Q₁, Ar³ and L3 are as defined and discussed above, and R₄ and R₅ independently represent hydrogen or one or more optional substituents. In this subclass, it is currently preferred that
A₁ is hydrogen,
Q₁ is hydrogen,
X₁ is —S—,
Ar³ is optionally substituted phenyl,
L3 is a bond, —O—, —S—, or —NR— wherein R is hydrogen or C₁-C₃ alkyl.

Particularly preferred in this subclass are compounds (IA) wherein A₁ is hydrogen, Q₁ is hydrogen, X₁ is —S—, Ar³ is optionally substituted phenyl and L3 is a bond. In this subclass, optional substituents R₄ and R₅ and optional substituents in Ar³ are preferably independently selected from fluoro, chloro, bromo, (C₁-C₃)alkyl, trifluoromethyl, (C₁-C₃)alkoxy, trifluoromethoxy, trifluoromethylthio, dimethylamino, cyano, (C₁-C₃alkyl)SO₂—, NH₂SO₂—, (C₁-C₃alkyl)NHSO₂—, (C₁-C₃alkyl)₂NSO₂—, —CONR^{A}R^{B}, and —NR^{B}COR^{A} wherein R^{A} and R^{B} are independently hydrogen or a (C₁-C₆)alkyl group, or R^{A} and R^{B} are linked to the same N atom to form a cyclic amino ring.

One preferred subclass of the compounds with which the invention is concerned consists of compounds of formula (IA), and salts, hydrates and solvates thereof:

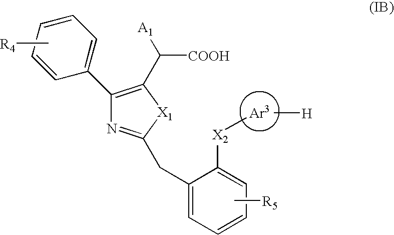

wherein A₁ is hydrogen or methyl, X₂ is a bond, —CH₂—, —O—, —S—, or —NR— wherein R is hydrogen or C₁-C₃ alkyl and X₁ and Ar³ are as defined in claim 1, and R₄ and R₅ independently represent hydrogen or one or more optional substituents. In this subclass, it is currently preferred that
A₁ is hydrogen,
X₁ is —S—,
Ar³ is optionally substituted phenyl,
X₂ is —CH₂— or a bond.

In this subclass, optional substituents R₄ and R₅ and optional substituents in Ar³ are independently selected from fluoro, chloro, bromo, (C₁-C₃)alkyl, trifluoromethyl, (C₁-C₃)alkoxy, trifluoromethoxy, trifluoromethylthio, dimethylamino, cyano, (C₁-C₃alkyl)SO₂—, NH₂SO₂— (C₁-C₃alkyl)NHSO₂—, (C₁-C₃alkyl)₂NSO₂—, —CONR^{A}R^{B}, and —NR^{B}COR^{A} wherein R^{A} and R^{B} are independently hydrogen or a (C₁-C₆)alkyl group, or R^{A} and R^{B} are linked to the same N atom to form a cyclic amino ring.

Specific examples of compounds with which the invention is concerned include:
[2-benzhydryl-4-(4-chlorophenyl)-thiazol-5-yl]-acetic acid,
[2-benzhydryl-4-(4-fluoro-phenyl)-thiazol-5-yl]-acetic acid,
[2-[1-(4-chloro-phenyl)-2-phenyl-ethyl]-4-(4-fluoro-phenyl)-thiazol-5-yl]-acetic acid,
{4-(4-chloro-phenyl)-2-[(4-chloro-phenyl)-phenyl-methyl]-thiazol-5-yl}-acetic acid,
[2-[(4-chloro-phenyl)-phenyl-methyl]-4-(4-fluoro-phenyl)-thiazol-5-yl]-acetic acid,
[2-[bis-(4-fluoro-phenyl)-methyl]-4-(4-fluoro-phenyl)-thiazol-5-yl]-acetic acid,
{4-(4-fluoro-phenyl)-2-[(4-methoxy-phenyl)-phenyl-methyl]-thiazol-5-yl}-acetic acid,
{4-(4-chloro-phenyl)-2-[(4-methoxy-phenyl)-phenyl-methyl]-thiazol-5-yl}-acetic acid,
[2-[(3,4-difluoro-phenyl)-phenyl-methyl]-4-(4-fluoro-phenyl)-thiazol-5-yl]-acetic acid,
[2-[bis-(4-methoxy-phenyl)-methyl]-4-(4-fluoro-phenyl)-thiazol-5-yl]-acetic acid,
[2-benzhydryl-4-(3-fluoro-phenyl)-thiazol-5-yl]-acetic acid,
[2-[bis-(4-fluoro-phenyl)-methyl]-4-(3,4-difluoro-phenyl)-thiazol-5-yl]-acetic acid,
[2-benzhydryl-4-(3,4-difluoro-phenyl)-thiazol-5-yl]-acetic acid,
[2-[bis-(4-fluoro-phenyl)-methyl]-4-(3-fluoro-phenyl)-thiazol-5-yl]-acetic acid,
   and salts hydrates and solvates thereof.

The invention also includes pharmaceutical compositions comprising a compound formula (II) or (IIA) together with a pharmaceutically acceptable carrier.

### Compositions

As mentioned above, the compounds with which the invention is concerned are capable of modulating CRTH2 activity, and are useful in the treatment of diseases which benefit from such modulation. Examples of such diseases are referred to above, and include asthma, rhinitis, allergic airway syndrome, and allergic rhinobronchitis.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial, as is required in the pharmaceutical art.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

For topical application to the eye, the drug may be made up into a solution or suspension in a suitable sterile aqueous or non aqueous vehicle. Additives, for instance buffers such as sodium metabisulphite or disodium edeate; preservatives including bactericidal and fungicidal agents such as phenyl mercuric acetate or nitrate, benzalkonium chloride or chlorhexidine, and thickening agents such as hypromellose may also be included.

The drug may also be formulated for inhalation, for example as a nasal spray, or dry powder or aerosol inhalers.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

The compounds with which the invention is concerned may be administered alone, or as part of a combination therapy with other drugs used for treatment of diseases with a major inflammatory component. In the case of asthma, rhinitis, and allergic airway syndrome such drugs include corticosteroids, long-acting inhaled beta-agonists, beta agonists, cromolyn, nedocromil, theophylline, leukotriene receptor antagonists, antihistamines, and anticholinergics (e.g. ipratropium), and are often administered as nasal sprays, dry powder or aerosol inhalers.

In the case of arthritis and related inflammatory diseases other known drugs include glucocorticoids, NSAIDs (Non Steroidal Anti-Inflammatory Drugs—conventional prostaglandin synthesis inhibitors, COX-2 inhibitors, salicylates), and DMARDs (disease-modifying anti-rheumatic drugs such as methotrexate, sulfasalazine, gold, cyclosporine).

### Synthetic Routes

There are multiple synthetic strategies for the synthesis of the compounds (I) with which the present invention is concerned, but all rely on known chemistry, known to the synthetic organic chemist. Thus, compounds according to formula (I) can be synthesised according to procedures described in the standard literature and are well-known to the one skilled in the art. Typical literature sources are “Advanced organic chemistry”, 4^{th} Edition (Wiley), J March, “Comprehensive Organic Transformation”, 2^{nd} Edition (Wiley), R. C. Larock, “Handbook of Heterocyclic Chemistry”, 2^{nd} Edition (Pergamon), A. R. Katritzky), review articles such as found in “Synthesis”, “Acc. Chem. Res.”, “Chem. Rev”, or primary literature sources identified by standard literature searches online or from secondary sources such as “Chemical Abstracts” or “Beilstein”.

In the following discussion of synthetic routes, the ring “Ar¹” is the ring shown in formula (I) containing X₁.

The linker L2 can be formed by joining two appropriately functionalised and, if needed, suitably protected fragments containing La2 and Lb2 as reactive moieties as outlined below. La2 and Lb2 are defined as any moieties that can react by e.g. a nucleophilic substitution, addition to multiple bonds or cyclisation reaction to form a given L2 linker as in:

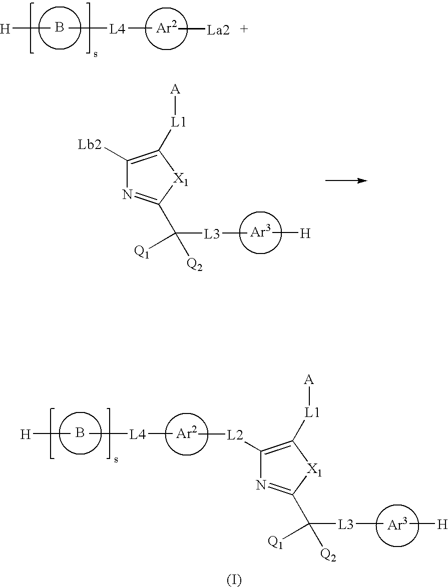

For example, the linker -L2- being -Alk¹-Z-(Alk²)ₚ- can be formed by reacting HBL4Ar²-Alk¹-“leaving group” with a nucleophilic derivative H-Z-(Alk²)ₚ-Ar¹(L1A)L3Ar³H wherein Z could be O, S or NR and Alk¹ could be an alkyl group. The reactions can also be made by reversing the functionalisation of La2 and Lb2 to make the connection between Z and Alk². The linkers having Z being SO or SO₂ can be obtained by oxidations of the corresponding -(Alk¹)ₘ-S-(Alk²)ₚ- derivatives during appropriate conditions.

Further representative examples, -L2- being -Alk¹-Z-(Alk²)ₚ- wherein Z is NH(CO) or NHSO₂ can be formed by reacting HBL4Ar²-(Alk¹)-NH₂ with an acylating derivative “leaving group”-CO-(Alk²)ₚ—Ar¹(L1A)L3Ar³H or “leaving group”-SO₂-(Alk²)ₚ-Ar¹(L1A)L3Ar³H, respectively. Alternatively, the conversion can be made directly with the acids HO—CO-(Alk²)ₚ—Ar¹(L1A)L3Ar³H and HO—SO₂-(Alk²)ₚ—Ar¹(L1A)L3ArH, respectively, using suitable coupling reagents such as dicyclohexylcarbodiimide (DCC), and promoters such as 1-hydroxybenzotriazole. Analogously, -L2- being -Alk¹-Z-(Alk²)ₚ- wherein Z being NH(CO)NH can be formed by reacting HBL4Ar²-(Alk¹)-NH₂ with an isocyanate derivative OCN-(Alk²)ₚ—Ar¹(L1A)L3Ar³H using suitable acid or base catalysis. The reactions can also be made by reversing the functionalisation of La2 and Lb2 to provide the “retro-bonds” in the case of NH(CO) or NHSO₂. Analogously, the connections can be made between Z and Alk².

Likewise, L2 being -(Alk¹)ₘ-Z-(Alk²)ₚ- wherein Z is a 5-membered heterocyclic system exemplified by

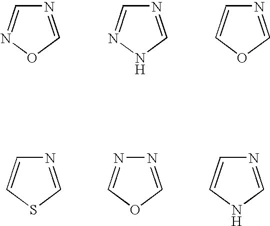

can be made according to standard cyclisation procedures using appropriate solvents, catalysts and temperatures. For example, formation of 1,2,4-triazole can be made with La2 being acylhydrazide and Lb2 being amide or thioamide or the reverse orientation of La2 and Lb2. 1,2,4-Oxadiazole can be formed from La2 being amidoxime and Lb2 being carboxylic ester or the reverse orientation of La2 and Lb2. 1,3,4-Oxadiazole can be formed from La2 being acylhydrazide and Lb2 being carboxylic ester or the reverse orientation of La2 and Lb2. The thiazole can be made from La2 being thioamide and Lb2 being an α-haloketone or the reverse orientation of La2 and Lb2.

In an analogous manner the compounds of formula (I) can be made by forming the linkers L3 or L4, according to procedures outlined for L2, as depicted below. Thus, La and Lb are defined as any moieties that can react by e.g. a nucleophilic substitution, addition to multiple bonds or cyclisation reaction to form a given linker L as exemplified below.

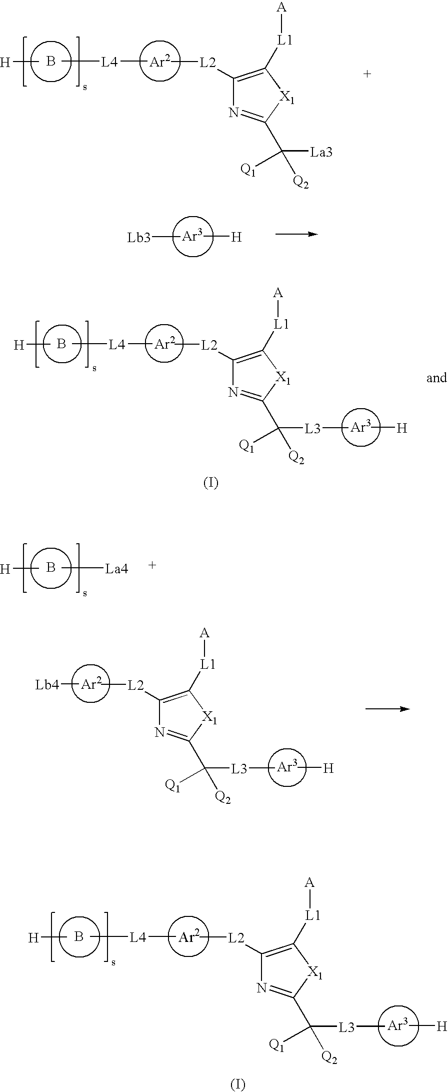

The Ar¹ moiety can also be the central scaffold that is used in connecting the L1, L2 and L3 parts in a stepwise fashion. This can be done via aromatic substitutions of the Ar¹ core to attach L1, L2 and/or L3, which then can be further functionalised to give the final Formula I compounds.

Furthermore, the five-membered X₁-azole moiety can also be assembled via conventional ring cyclisation reactions with reactants containing the L1, L2 and L3 units either containing the full appendices as exemplified below wherein Lg is a leaving group such as halogen,

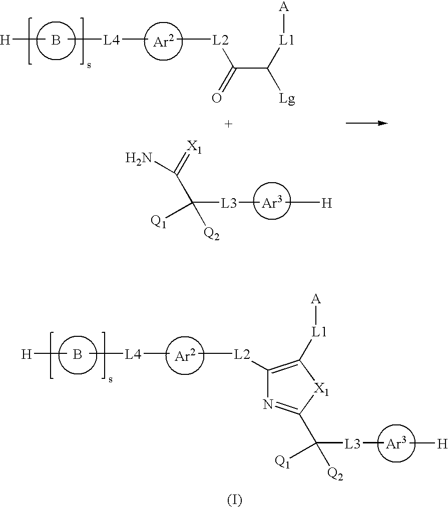

or in forms that can be further functionalised into the final formula (I) structures as described previously. One such illustration is given below:

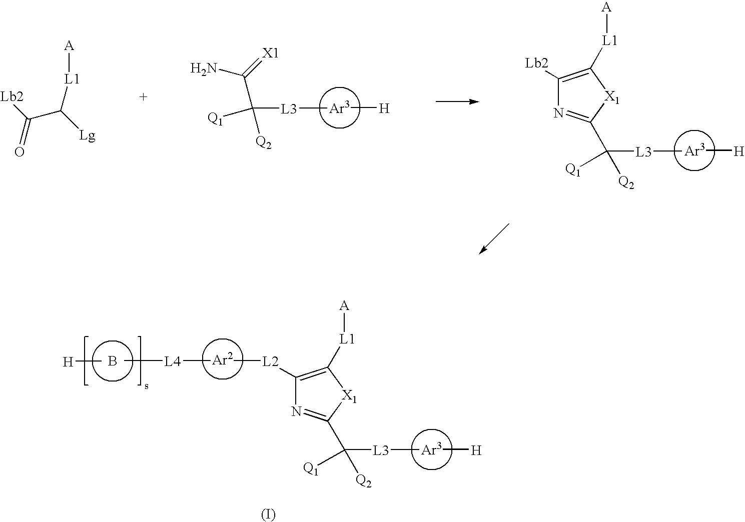

For example, 1,2,4-triazoles can be made from acylhydrazines and amides or thioamides; 1,2,4-oxadiazoles from amidoximes and carboxylic esters; 1,3,4-oxadiazoles from acylhydrazines and carboxylic esters; thiazoles from thioamides and α-haloketones; pyrazines, pyrimidines and pyridines via various condensation and cycloaddition reactions.

The building blocks used in the reactions are either commercially available or made according to standard procedures well-know to one skilled in the art as described in “Advanced organic chemistry”, 4^{th} Edition (Wiley), J March, “Comprehensive Organic Transformation”, 2^{nd} Edition (Wiley), R. C. Larock, “Handbook of Heterocyclic Chemistry”, 2^{nd} Edition (Pergamon), A. R. Katritzky or other suitable literature sources.

The Examples herein describe specific strategies for the synthesis of compounds (I) wherein X1 is S.

The following Examples illustrate the preparation of compounds with which this invention is concerned. Some compounds were synthesised, and some were acquired from commercial sources. In the Examples:

### General Comments:

NMR spectra were obtained on a Bruker Avance AMX 300 MHz instrument. For some compounds only selected characteristic ¹H NMR signals are reported. LC/MS was performed on an Agilent 1100-series instrument. LC/MS methods are as follows: An10p8: Column: XTerra MS C18; Flow: 1.0 mL/min; Gradient: 0-5 min: 15-100% MeCN in water, 5-72 min: 100% MeCN; Modifier: 5 mM ammonium formate; MS-ionisation mode: API-ES (pos.). An10n8: Column: XTerra MS C18; Flow: 1.0 mL/min; Gradient: 0-5 min: 15-100% MeCN in water, 5-7½ min: 100% MeCN; Modifier: 5 mM ammonium formate; MS-ionisation mode: API-ES (neg.).

### General Synthetic Route I

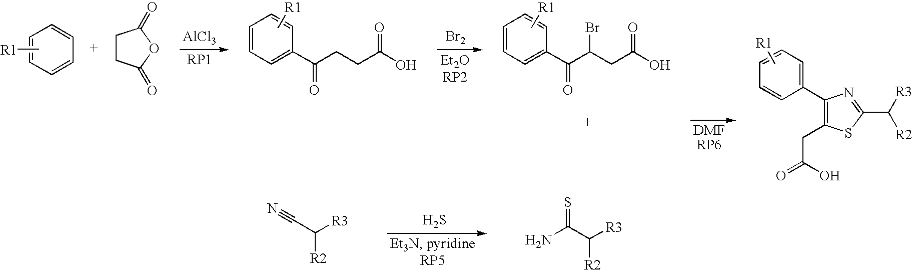

### Synthetic Routes to Nitrile Intermediates:

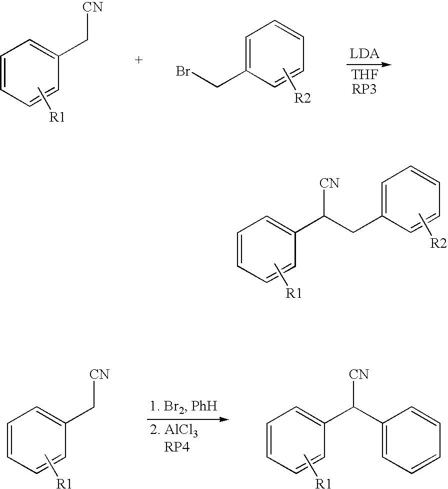

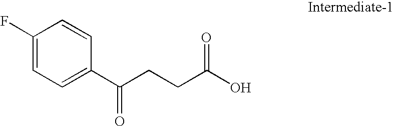

4-(4-Fluoro-phenyl)-4-oxo-butyric acid—Representative Procedure 1a (RP1a): Succinic anhydride (1.0 g, 10 mmol) was dissolved in 4-fluorobenzene (3.75 ml, 40 mmol) and cooled to −9° C. under nitrogen. AlCl₃ (2.67 g; 20 mmol) was added, and the temperature was kept between −9 and 0° C. for 4½ hours. The reaction was allowed to warm to room temperature and stirred over night. The reaction mixture was poured into aqueous 4 M HCl (10 ml) at 0° C., and the precipitated was filtered and washed with water. The solid was recrystallized from toluene to give 1.40 g (71%) of a colorless solid: LC/MS (an10n8): Rt 0.26 min, m/z 195 [M−H], 413 [2M−2H+Na]; ¹H NMR (CDCl₃): δ 2.84 (t, J=6.5 Hz, 2H), 3.31 (t, J=6.5 Hz, 2H), 7.16 (m, 3H), 8.03 (m, 2H).

In an analogous way, the following compounds were made:

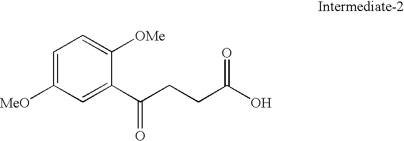

4-(2,5-Dimethoxy-phenyl)-4-oxo-butyric acid. LC/MS (an10n8): Rt 0.50 min, m/z 237 [M−H], 497 [2M−2H+Na]; ¹H NMR (DMSO-d₆): δ 2.53 (t, J=6.0 Hz, 2H), 3.16 (t, J=6.3 Hz, 2H), 3.73 (s, 3H), 3.85 (s, 3H), 7.12-7.14 (m, 3H). ¹³C NMR/APT (DMSO-d₆): δ 29.1 (CH2), 39.2 (CH2), 56.4 (CH3), 57.2 (CH3), 114.5 (CH), 115.0 (CH), 120.4 (CH), 128.5 (C), 153.8 (C), 174.7 (CO), 200.2 (CO).

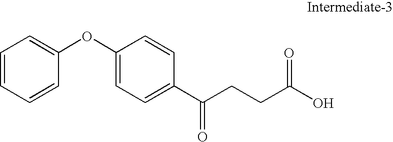

4-Oxo-4-(4-phenoxy-phenyl)-butyric acid. ¹H NMR (DMSO-d₆): δ 2.57 (t, J=6.22, 2H), 3.21 (t, J=6.22, 2H), 7.05 (m, 2H), 7.13 (m, 2H), 7.25 (m, 1H), 7.47 (m, 2H), 8.01 (m, 2H), 12.13 (brs, 1H (COOH)).

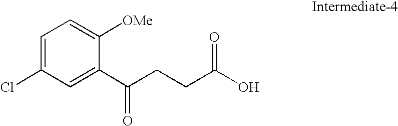

4-(5-Chloro-2-methoxy-phenyl)-4-oxo-butyric acid. ¹H NMR (DMSO-d6): δ 2.53 (t, J=6.6 Hz, 2H), 3.15 (t, J=6.0 Hz, 2H), 3.90 (s, 3H), 7.23 (d, J=8.9 Hz, 1H), 7.53 (d, J=2.6 Hz, 1H), 7.60 (dd. J=2.9, 8.9 Hz, 1H) and 12.11 (br s, 1H).

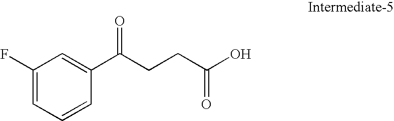

4-(3-Fluoro-phenyl)-4-oxo-butyric acid—Representative Procedure 1b (RP1): In a flame dried flask under nitrogen, succinic anhydride (471 mg, 4.7 mmol) was dissolved in dry THF (5 mL). The mixture was cooled to −78° C. 3-Fluorophenyl-magnesium bromide in THF (1N, 5 mL, 5 mmol) was added slowly. The mixture was stirred at −78° C. for 3 h, then allowed to raise room temperature. The mixture was transferred into 1N HCl (aq.) then extracted with CH₂Cl₂. The organic layer was dried and evaporated. The residue was purified by flash chromatography on SiO₂ to give 350 mg (38%) of the title compound: ¹H NMR (CDCl₃): δ 2.84 (t, J=6.5 Hz, 2H), 3.31 (t, J=6.5 Hz, 2H), 7.30 (m, 1H), 7.47 (td, J=8.1, 5.7, 1H), 7.68 (m, 1H), 7.79 (m, 1H)

The following compounds were prepared by an analogous procedure:

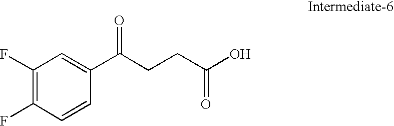

4-(3,4-Difluoro-phenyl)-4-oxo-butyric acid. ¹H NMR (CDCl₃): δ 2.57 (t, J=6.2 Hz, 2H), 3.25 (t, J=6.2 Hz, 2H), 7.60 (m, 1H), 7.87 (m, 1H), 8.02 (m, 1H), 12.17 (br s, 1H).

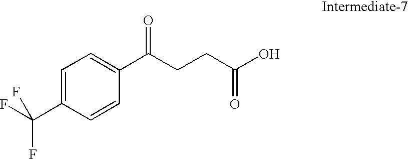

4-Oxo-4-(4-trifluoromethyl-phenyl)-butyric acid. 4-Iodobenzotrifluoride (500 μL, 3.4 mmol) was dissolved in dry diethylether (5 mL) in a flame dried flask under nitrogen. Magnesium (83 mg, 3.4 mmol) was added and the mixture was stirred at room temperature for 45 min. Succinic anhydride was dissolved in THF (5 mL) in a flame dried flask under nitrogen then cooled to −78° C. The freshly made Grignard reagent was added slowly. The mixture was allowed to warm to room temperature over 3 h. The mixture was transferred into NH₄Cl (aq.) then extracted with CH₂Cl₂. The organic layer was dried (MgSO₄) then evaporated. The product was purified by flash chromatography on SiO₂ to give 250 mg (30%): ¹H NMR (CDCl₃): δ 2.86 (t, J=6.4 Hz, 2H), 3.35 (t, J=6.4 Hz, 2H), 7.76 (d, J=8.3 Hz, 2H), 8.10 (d, J=8.3 Hz, 2H).

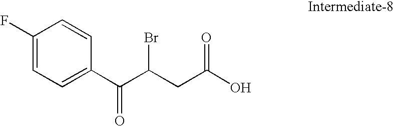

3-Bromo-4-(4-fluoro-phenyl)-4-oxo-butyric acid—Representative Procedure 2 (PR2): 4-(4-Fluoro-phenyl)-4-oxo-butyric acid (1.20 g, 6.12 mmol) was suspended in ether (10 mL) at room temperature. Bromine (0.34 ml, 6.73 mmol) was added drop-wise. After 4 hours, the reaction was concentrated and the residue was recrystallized from ether/heptane to give 1.26 g (75%) of pale orange crystals. ¹H NMR (CDCl₃): δ 3.16 (dd, J=2.2, 6.9 Hz, 1H), 3.56 (dd, J=3.5, 6.9 Hz, 1H), 5.41 (dd, J=2.2, 3.5 Hz, 1H), 7.19 (m, 2H), 8.08 (m, 2H); ¹³C NMR/APT (CDCl₃): δ 190.9, 175.6, 168.3, 164.9, 132.3, 132.1, 116.6, 116.3, 38.8, 38.3.

In an analogous way, the following compounds were made:

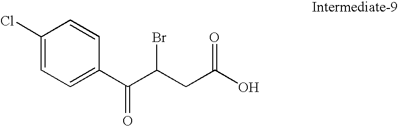

3-Bromo-4-(4-chloro-phenyl)-4-oxo-butyric acid. ¹H NMR (CDCl₃, 300 MHz): δ 3.18 (dd, J=5.7, 17.5 Hz, 1H), 3.52 (dd, J=8.5, 17.5 Hz, 1H), 5.43 (dd, J=5.8, 8.5 Hz, 1H), 6.99 (d, J=8.9 Hz, 2H), 8.10 (d, J=8.9 Hz, 2H).

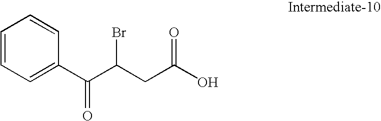

3-Bromo-4-oxo-4-phenyl-butyric acid. LC/MS (an10p8): Rt 2.69 min, m/z 257/259 [M+H], 279/281 [M+Na]; ¹H NMR (CDCl₃): δ 3.17 (dd, J=5.7, 17.6 Hz, 1H), 3.56 (dd, J=8.7, 17.6 Hz, 1H), 5.46 (dd, J=5.7, 8.7 Hz, 1H), 7.52 (m, 2H), 7.64 (m, 1H), 8.05 (m, 2H).

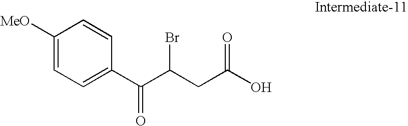

3-Bromo-4-(4-methoxy-phenyl)-4-oxo-butyric acid. ¹H NMR (CDCl₃): δ 3.17 (dd, J=5.7, 17.7 Hz, 1H), 3.55 (dd, J=9.0, 17.7 Hz, 1H), 3.91 (s, 3H), 5.40 (dd, J=5.5, 8.9 Hz, 1H), 7.51 (d, J=8.7 Hz, 2H), 7.98 (d, J=8.7 Hz, 2H).

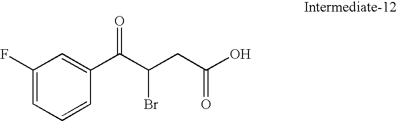

3-Bromo-4-(3-fluoro-phenyl)-4-oxo-butyric acid. ¹H NMR (CDCl₃): δ 3.18 (m, 1H), 3.54 (m, 1H), 5.39 (m, 1H), 7.34 (m, 1H), 7.50 (m, 1H), 7.83 (m, 2H) and 10.11 (br s, 1H).

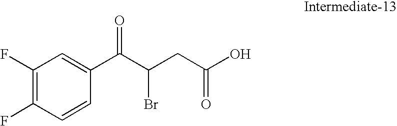

3-Bromo-4-(3,4-difluoro-phenyl)-4-oxo-butyric acid. ¹H NMR (CDCl₃): δ 3.16 (dd, J=17.7, 5.3 Hz, 1H), 3.55 (dd, J=17.7, 9.0 Hz, 1H), 5.32 (dd, J=9.0, 5.5 Hz, 1H), 7.31 (m, 1H), 7.85 (m, 2H).

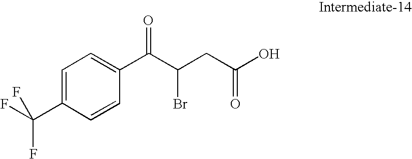

3-Bromo-4-oxo-4-(4-trifluoromethyl-phenyl)-butyric acid. ¹H NMR (CDCl₃): δ 3.19 (dd, J=17.7, 5.5 Hz, 1H), 3.59 (dd, J=17.5, 9.0 Hz, 1H), 5.44 (dd, J=9.0, 5.5 Hz, 1H), 7.79 (d, J=2.8 Hz) and 8.16 (d, J=3.0 Hz).

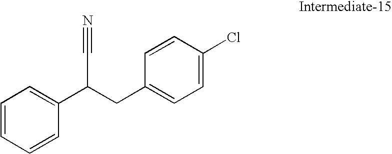

3-(4-Chloro-phenyl)-2-phenyl-propionitrile—Representative Procedure 3 (RP3): The reaction was preformed under N₂ in flame dried glassware. Benzylcyanide (1.2 mL, 10 mmol) was added slowly (over a period of 40 min) to a solution of LDA (10 mmol) in THF (10 mL) at −78° C. The mixture was stirred for 1 h at −78° C., and then added slowly (over a period of 16 min) to a solution of 4-chlorobenzyl bromide in THF (10 mL) at −78° C. The mixture was stirred for 1 h at −78° C. and left over night to reach room temperature. The reaction mixture was added saturated aq. NH₄Cl and the aqueous mixture was extracted with EtOAc. The organic phase was washed with brine, dried (MgSO₄) and concentrated, and the residue was recrystallized from heptane to give 1.26 g (53%) of a light brown powder. ¹H NMR (CDCl₃): δ 3.16 (m, 2H) and 4.01 (t, J=7.2 Hz, 1H), 7.05 (d, J=8.3 Hz, 2H), 7.32 (m, 7H).

In an analogous way, the following compounds were made:

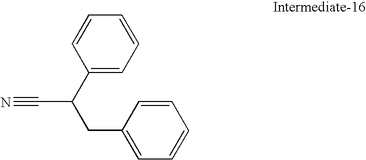

2,3-Diphenyl-propionitrile. ¹H NMR (CDCl₃): δ 3.19 (m, 2H) and 4.02 (t, J=7.9 Hz, 1H).

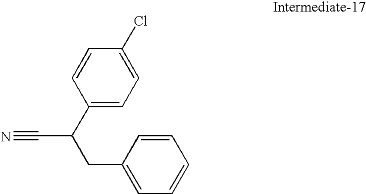

2-(4-Chloro-phenyl)-3-phenyl-propionitrile. ¹H NMR (CDCl₃): δ 3.16 (m, 2H) and 4.14 (t, J=7.2 Hz, 1H).

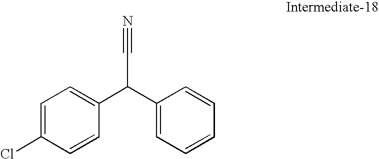

(4-Chloro-phenyl)-phenyl-acetonitrile—Representative Procedure 4 (RP4): The reaction was preformed under N₂ and flame dried glassware. 4-Chlorobenzyl cyanide (1 mL; 7.8 mmol) was dissolved in benzene (10 mL) and heated to reflux. Bromide (442 μL; 8.6 mmol) was added over 30 min. The mixture was stirred 20 min at reflux, then cooled to approx 40° C. and added over 30 min to a refluxing mixture of aluminum chloride (1 g; 7.8 mmol) in benzene (10 mL). The mixture was stirred 1 h at reflux then cooled to r.t. The mixture was transferred into ice and conc. HCl (50 mL) then extracted with diethyl ether. The organic layer was dried (MgSO₄) and concentrated in vacuum to give brown oil. The product was purified by flash chromatography on SiO₂ to give 523 mg (28%) yellow oil. ¹H NMR (CDCl₃): δ 5.14 (s, 1H) and 7.36 (m, 9H). APT (CDCl₃): δ 42.42 (CH).

In an analogous way, the following compounds were made:

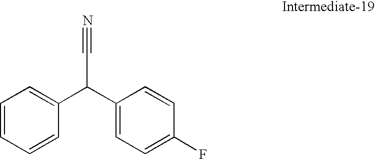

(4-Fluoro-phenyl)-phenyl-acetonitrile. ¹H NMR (CDCl₃): δ 5.15 (s, 1H), 7.08 (m, 2H), 7.36 (m, 7H).

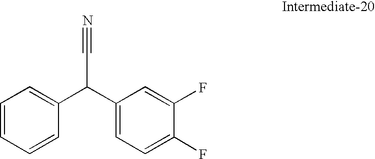

(3,4-Difluoro-phenyl)-phenyl-acetonitrile. ¹H NMR (CDCl₃, 300 MHz): δ 5.12 (s, 1H), 7.15 (m, 3H), 7.41 (m, 5H).

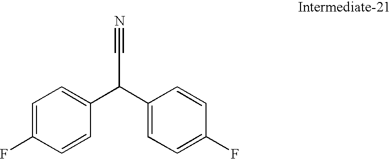

Bis-(4-fluoro-phenyl)-acetonitrile. 4,4′-Difluorobenzhydrol (1 g, 4.54 mmol) was dissolved in TFA (10 mL). Potassium cyanide (620 mg, 9.53 mmol) was added and the mixture was cooled to 0° C. Sulfuric acid (conc., 3 mL) was added slowly. The mixture was stirred at room temperature for 5 h then quenched with H₂O and ethyl acetate. The organic phase was washed with water and brine, dried (MgSO₄) and concentrated. The residue was purified by flash chromatography on SiO₂ to give 450 mg (43%) of the title compound: ¹H NMR (CDCl₃): δ 5.14 (s, 1H), 7.09 (m, 4H) and 7.32 (m, 4H). ¹³C NMR (APT, CDCl₃): δ 41.53 (CH).

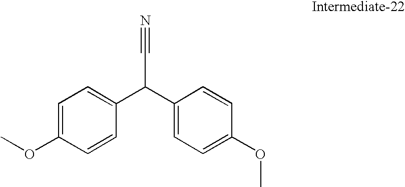

Bis-(4-methoxy-phenyl)-acetonitrile. 4,4′-Dimethoxybenzhydrol (5 g, 20 mmol) was dissolved in dry CH₂Cl₂ (50 mL) then cooled to 0° C. Thionylchloride (1.5 mL, 20 mmol) was added slowly. The mixture was stirred at room temperature for 4 h. The mixture was concentrated under vacuum. The product was dissolved in dry CH₂Cl₂ (10 mL) and added to a mixture of potassium cyanide (2.6 g, 40 mmol) and 18-crown-6 (500 mg) dissolved in dry CH₂Cl₂ (20 mL). The mixture was stirred over night at room temperature. The mixture was transferred into water then extracted with CH₂Cl₂. The organic layer was washed with water and brine, dried (MgSO₄) and concentrated. The residue was recrystallized from ethyl acetate to give 2.54 g (50%) of the title compound: ¹H NMR (CDCl₃): δ 3.82 (s, 6H), 5.07 (s, 1H), 6.90 (m, 4H), 7.25 (m, 4H).

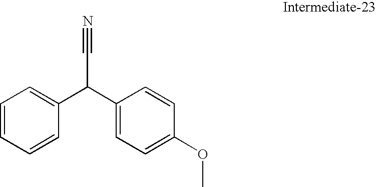

(4-Methoxy-phenyl)-phenyl-acetonitrile. (4-Methoxyphenyl)acetonitrile (5 mL, 37 mmol) was dissolved in dry benzene (10 mL) in a flame dried flask under nitrogen. The mixture was heated to reflux and bromine (1.9 mL, 37 mmol) was added in small portions over 2 h. The mixture was stirred at reflux for 30 min, then added to a refluxing mixture of AlCl₃ (4.9 g, 37 mmol) in benzene (30 mL) over 80 min. The mixture was stirred at reflux for 1 h then cooled to room temperature. The mixture was transferred into 1N HCl and ice then extracted with diethylether. The organic layer was dried (MgSO₄) and concentrated. The product was purified by flash chromatography on SiO₂ to give 1.3 g (16%) of the title compound: ¹H NMR (CDCl₃): δ 3.82 (s, 3H), 5.12 (s, 1H), 6.90 (m, 2H), 7.27 (m, 2H), 7.37 (m, 5H).

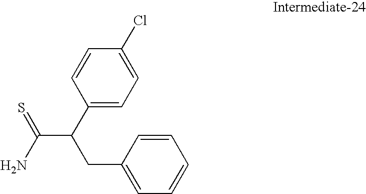

2-(4-Chloro-phenyl)-3-phenyl-thiopropionamide—Representative Procedure 5 (RP5): 2-(4-Chloro-phenyl)-3-phenyl-propionitrile (200 mg, 0.8 mmol) was dissolved in pyridine (5 mL) and triethylamine (1 mL). The mixture was saturated with hydrogen sulfide and stirred under an atmosphere of hydrogen sulfide room temperature for 3 days. The mixture was extracted with EtOAc. The extract was washed with brine, dried (MgSO₄) and concentrated to give 250 mg of crude product, which was used directly in the next step. ¹H NMR (DMSO-d6): δ 3.17 (dd, J=8.1, 13.8 Hz, 1H), 3.75 (dd, J=7.0, 13.8 Hz, 1H), 4.03 (t, J=7.5 Hz, 1H) and 7.20 (m, 9H).

In an analogous way, the following compounds were made:

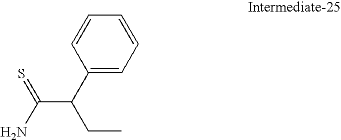

2-Phenyl-thiobutyramide. LC/MS (an10p8): Rt 2.9 min, m/z 180 [M+1]. ¹H NMR (CDCl₃): δ 0.93 (t, 3H); 2.01 (m, 1H); 2.41 (m, 1H); 3.75 (dd, 1H); 6.75 (br s, 1H); 7.36 (m, 5H); 7.58 (br s, 1H).

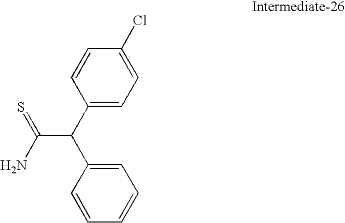

2-(4-Chloro-phenyl)-2-phenyl-thioacetamide. ¹H NMR (CDCl₃): δ 5.59 (s, 1H).

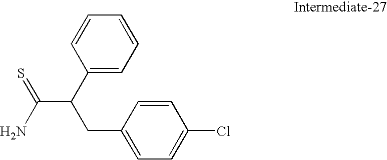

3-(4-Chloro-phenyl)-2-phenyl-thiopropionamide. ¹H NMR (DMSO-d₆): δ 3.14 (m, 1H), 3.77 (dd, J=6.6, 13.9 Hz, 1H), 4.00 (t, J=7.73 Hz, 1H).

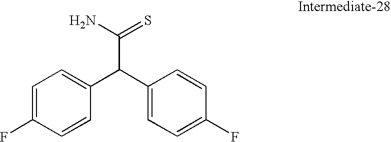

2,2-Bis-(4-fluoro-phenyl)-thioacetamide. ¹H NMR (CDCl₃): δ 5.57 (s, 1H), 6.76 (br s, 1H (NH)), 7.08 (m, 4H) and 7.23 (m, 4H) and 7.68 (br s, 1H (NH)).

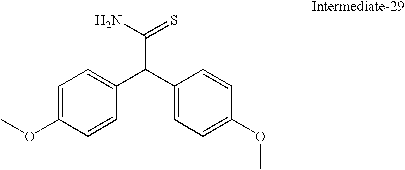

2,2-Bis-(4-methoxy-phenyl)-thioacetamide. LC/MS (an10p8): Rt 2.96 min, m/z 288 [M+H]; ¹H NMR (CDCl₃): δ 3.81 (s, 6H), 5.54 (s, 1H), 6.83 (bs, 1H (NH)), 6.90 (m, 4H), 7.18 (m, 4H), 7.73 (brs, 1H (NH)).

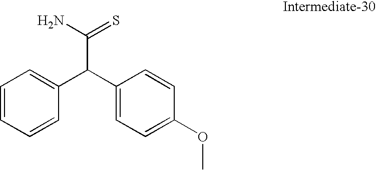

2-(4-Methoxy-phenyl)-2-phenyl-thioacetamide. ¹H NMR (CDCl₃): δ 3.82 (s, 3H), 5.59 (s, 1H), 6.83 (bs, 1H (NH)), 6.90 (m, 2H), 7.18 (m, 2H), 7.35 (m, 5H), 7.76 (bs, 1H (NH)).

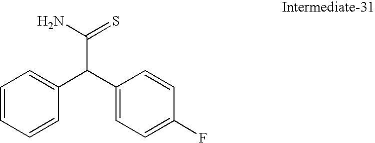

2-(4-Fluoro-phenyl)-2-phenyl-thioacetamide. ¹H NMR (CDCl₃): δ 5.61 (s, 1H), 6.84 (br s, 1H (NH)), 7.06 (m, 2H), 7.26 (m, 2H), 7.37 (m, 5H), 7.86 (br s, 1H (NH)).

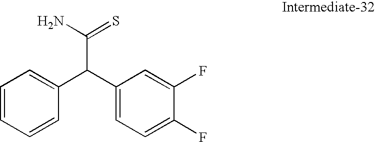

2-(3,4-Difluoro-phenyl)-2-phenyl-thioacetamide. ¹H NMR (CDCl₃): δ 5.56 (s, 1H), 6.81 (br s, 1H (NH)), 7.04 (m, 1H), 7.14 (m, 2H), 7.25 (m, 2H), 7.38 (m, 3H), 7.92 (br s, 1H (NH)).

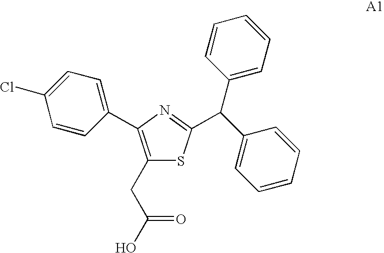

[2-Benzhydryl-4-(4-chloro-phenyl)-thiazol-5-yl]-acetic acid—Representative Procedure 6 (RP6): 3-Bromo-4-(4-chlorophenyl)-4-oxo-butyric acid (119 mg, 0.4 mmol) and 2,2-diphenyl-thioacetamide (91 mg, 0.4 mmol) was dissolved in DMF (1 mL) and heated to 100° C. for 10 minutes in a microwave oven. The reaction mixture was poured into water at 0° C. The precipitation was filtered off and recrystallized from CH₂Cl₂ to give 77 mg (54%) yellow powder: ¹H NMR (DMSO-d6): δ 3.91 (s, 2H), 5.96 (s, 1H), 7.37 (m, 10H), 7.52 (d, J=8.48 Hz, 2H), 7.60 (d, J=8.67 Hz, 2H), 12.82 (br s, 1H (COOH)).

In an analogous way, the following compounds were made:

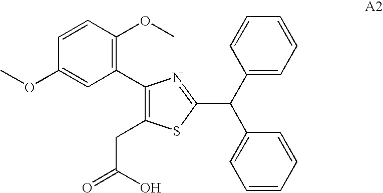

[2-Benzhydryl-4-(2,5-dimethoxy-phenyl)-thiazol-5-yl]-acetic acid. LC/MS (an10p8): Rt 3.18 min, m/z 446 [M+1]; ¹H NMR (DMSO-d₆): δ 3.59 (s, 2H), 5.92 (s, 1H), 6.85 (d, J=3.0 Hz, 1H), 6.96 (dd, J=8.9 and 3.0 Hz), 7.04 (d, J=8.5 Hz), 7.22-7.45 (m, 10H).

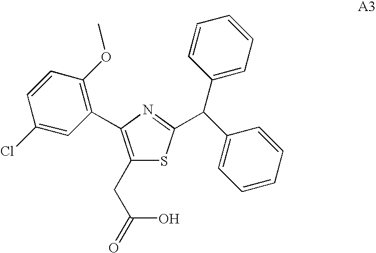

[2-Benzhydryl-4-(5-chloro-2-methoxy-phenyl)-thiazol-5-yl]-acetic acid. LC/MS (an10p8): Rt 3.70 min, m/z 450 [M+1]; ¹H NMR (DMSO-d₆): δ 3.60 (s, 2H), 3.73 (s, 3H), 5.93 (s, 1H), 7.14 (d, J=Hz, 1H), 7.28 (m, 3H), 7.36 (m, 8H), 7.44 (m, 1H).

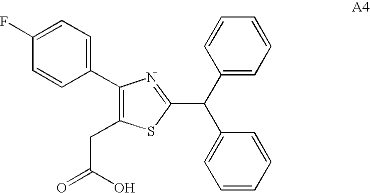

[2-Benzhydryl-4-(4-fluoro-phenyl)-thiazol-5-yl]-acetic acid. LC/MS (an10p8): Rt 2.93 min, m/z 404 [M+1]; ¹H NMR (CDCl₃): δ 3.85 (s, 2H), 5.88 (s, 1H), 7.12 (m, 2H), 7.25-7.38 (m, 10H), 7.58 (m, 2H).

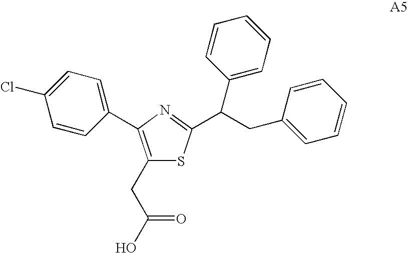

[4-(4-Chloro-phenyl)-2-(1,2-diphenyl-ethyl)-thiazol-5-yl]-acetic acid. LC/MS (an10p8): Rt 4.74 min, m/z 434 [M+1]; ¹H NMR (DMSO-d₆): δ 3.32 (m, 1H), 3.61 (m, 1H), 3.85 (s, 2H(CH₂)), 4.75 (m, 1H (CH)).

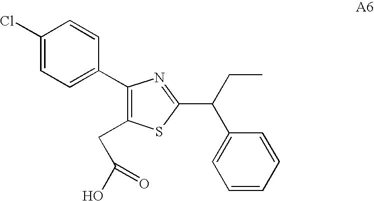

[4-(4-Chloro-phenyl)-2-(1-phenyl-propyl)-thiazol-5-yl]-acetic acid: LC/MS (an10p8): Rt 3.1 min, m/z 372 [M+1].

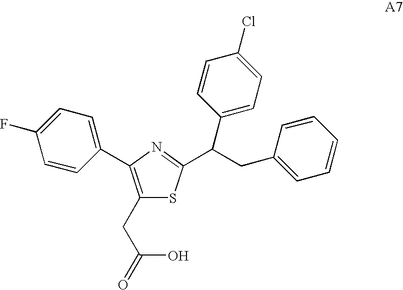

[2-[1-(4-Chloro-phenyl)-2-phenyl-ethyl]-4-(4-fluoro-phenyl)-thiazol-5-yl]-acetic acid. ¹H NMR (DMSO-d₆): δ 3.32 (m, 1H), 3.61 (m, 1H), 3.84 (s, 2H(CH₂)), 4.80 (m, 1H (CH)).

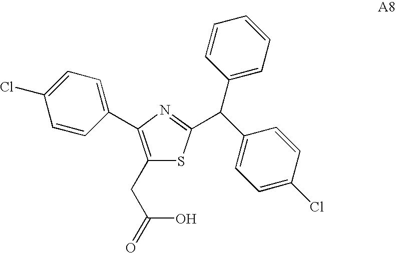

{4-(4-Chloro-phenyl)-2-[(4-chloro-phenyl)-phenyl-methyl]-thiazol-5-yl}-acetic acid. ¹H NMR (DMSO-d6): δ 3.91 (s, 2H), 6.01 (s, 1H), 7.37 (m, 9H), 7.50 (d, J=8.10 Hz, 2H), 7.60 (d, J=8.29 Hz, 2H).

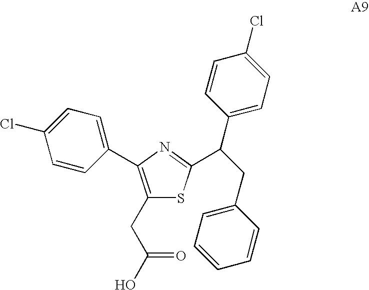

{4-(4-Chloro-phenyl)-2-[1-(4-chloro-phenyl)-2-phenyl-ethyl]-thiazol-5-yl}-acetic acid. LC/MS (an10p8): Rt 5.17 min, m/z 469 [M+1]; ¹H NMR (DMSO-d₆): δ 3.32 (m, 1H), 3.61 (dd, J=6.90 and 13.94, 1H), 3.86 (s, 2H(CH₂)) and 4.81 (t, J=7.90, 1H (CH)).

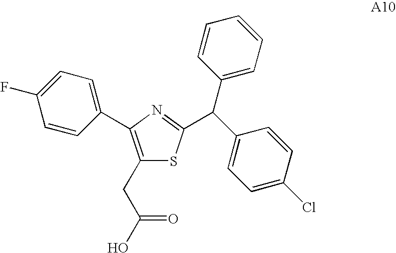

[2-[(4-Chloro-phenyl)-phenyl-methyl]-4-(4-fluoro-phenyl)-thiazol-5-yl]-acetic acid. LC/MS (an10p8): Rt 5.04 min, m/z 438 [M+1]; ¹H NMR (DMSO-d₆): δ3.83 (s, 2H), 5.86 (s, 1H), 7.11 (m, 2H), 7.20-7.40 (m, 9H), 7.55 (m, 2H).

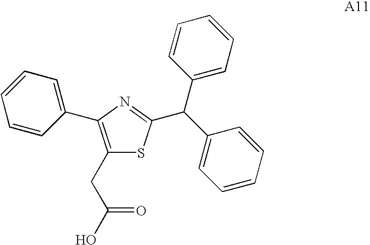

(2-Benzhydryl-4-phenyl-thiazol-5-yl)-acetic acid. LC/MS (an10p8): Rt 3.95 min, m/z 386 [M+H]; ¹H NMR (CDCl₃): δ 3.85 (s, 2H), 6.01 (s, 1H), 7.22-7.48 (m, 12H) and 7.60 (m, 2H).

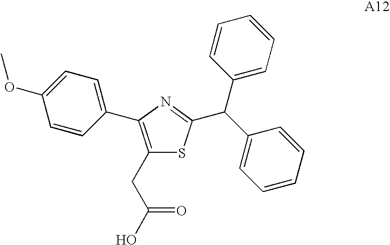

[2-Benzhydryl-4-(4-methoxy-phenyl)-thiazol-5-yl]-acetic acid. LC/MS (an10p8): Rt 3.52 min, m/z 416 [M+H]; ¹H NMR (CDCl₃): δ 3.84 (s, 3H), 3.85 (s, 2H), 5.86 (s, 1H), 6.97 (m, 2H), 7.26-7.37 (m, 10H) and 7.54 (m, 2H).

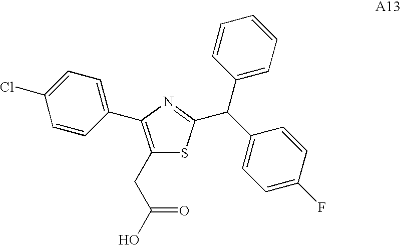

{4-(4-Chloro-phenyl)-2-[(4-fluoro-phenyl)-phenyl-methyl]-thiazol-5-yl}-acetic acid. LC/MS (an10p8): Rt 0.98 min, m/z 438 [M+H]; ¹H NMR (DMSO-d₆): δ 3.91 (s, 2H), 6.00 (s, 1H), 7.19 (m, 2H), 7.29 (m, 1H), 7.36-7.43 (m, 6H), 7.54 (m, 2H), 7.59 (m, 2H), 12.87 (br s, 1H).

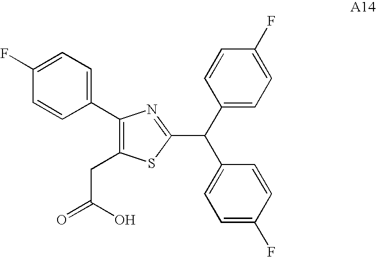

[2-[Bis-(4-fluoro-phenyl)-methyl]-4-(4-fluoro-phenyl)-thiazol-5-yl]-acetic acid. LC/MS (an10p8): Rt 4.81 min, m/z 440 [M+H]; ¹H NMR (DMSO-d₆): δ 3.89 (s, 2H), 6.03 (s, 1H), 7.19 (m, 4H), 7.30 (m, 2H), 7.41 (m, 4H), 7.62 (m, 2H).

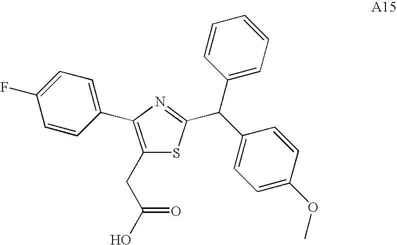

{4-(4-Fluoro-phenyl)-2-[(4-methoxy-phenyl)-phenyl-methyl]-thiazol-5-yl}-acetic acid. LC/MS (an10p8): Rt 3.45 min, m/z 434 [M+H]; ¹H NMR (CDCl₃): δ 3.81 (s, 3H), 3.84 (s, 2H), 5.81 (s, 1H), 6.89 (m, 2H), 7.12 (m, 2H), 7.24 (m, 2H), 7.28-7.38 (m, 5H), 7.58 (m, 2H). ¹³C-APT (CDCl₃): δ 32.79 (CH₂), 54.62, 55.65 (CH₃/CH).

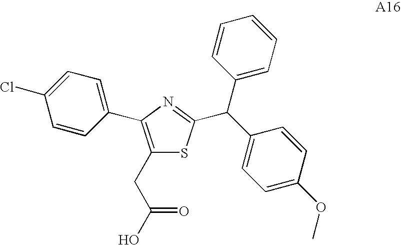

{4-(4-Chloro-phenyl)-2-[(4-methoxy-phenyl)-phenyl-methyl]-thiazol-5-yl}-acetic acid. LC/MS (an10p8): Rt 3.71 min, m/z 450 [M+H]; ¹H NMR (CDCl₃): δ 3.81 (s, 3H), 3.84 (s, 2H), 5.84 (s, 1H), 6.87 (m, 2H), 7.21 (m, 2H), 7.24-7.36 (m, 5H), 7.41 (m, 2H), 7.55 (m, 2H). ¹³C-APT (CDCl₃): δ 32.83 (CH₂), 54.57, 55.66 (CH₃/CH).

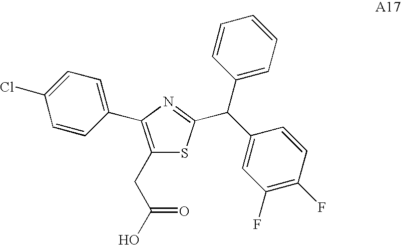

{4-(4-Chloro-phenyl)-2-[(3,4-difluoro-phenyl)-phenyl-methyl]-thiazol-5-yl}-acetic acid. LC/MS (an10p8): Rt 3.98 min, m/z 456 [M+H]; ¹H NMR (CDCl₃): δ 3.89 (s, 2H), 5.79 (s, 1H), 7.04 (m, 1H), 7.15 (m, 2H), 7.25-7.40 (m, 5H), 7.44 (m, 2H), 7.53 (m, 2H). ¹³C-APT (CDCl₃): δ 32.60 (CH₂), 54.54 (CH).

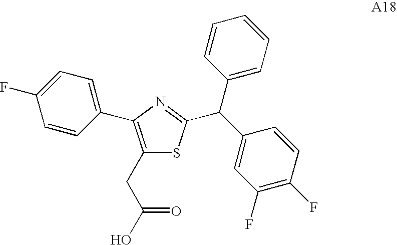

[2-[(3,4-Difluoro-phenyl)-phenyl-methyl]-4-(4-fluoro-phenyl)-thiazol-5-yl]-acetic acid. LC/MS (an10p8): Rt 3.76 min, m/z 440 [M+H]; ¹H NMR (CDCl₃): δ 3.88 (s, 2H), 5.83 (s, 1H), 7.03 (m, 1H), 7.14 (m, 4H), 7.28-7.44 (m, 5H), 7.58 (m, 2H). ¹³C-APT (CDCl₃): δ 32.65 (CH₂), 54.46 (CH).

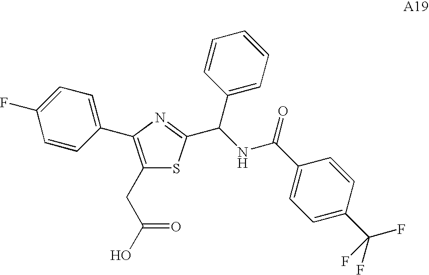

{4-(4-Fluoro-phenyl)-2-[phenyl-(4-trifluoromethyl-benzoylamino)-methyl]-thiazol-5-yl}-acetic acid. LC/MS (an10p8): Rt 2.88 min, m/z 515 [M+H]; ¹H NMR (DMSO-d₆): δ 3.90 (s, 2H), 6.64 (d, J=8.2 Hz, 1H), 7.30 (m, 2H), 7.36-7.45 (m, 3H), 7.54-7.65 (m, 4H), 7.87 (d, J=8.2 Hz, 2H), 8.16 (d, J=8.2 Hz, 2H), 9.87 (m, 1H (NH)) and 12.82 (s, 1H (OH)).

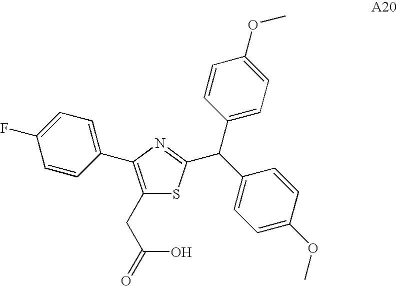

[2-[Bis-(4-methoxy-phenyl)-methyl]-4-(4-fluoro-phenyl)-thiazol-5-yl]-acetic acid. LC/MS (an10p8): Rt 2.17 min, m/z 464 [M+H]; ¹H NMR (CDCl₃): δ 3.81 (s, 6H), 3.83 (s, 2H), 5.77 (s, 1H), 6.89 (m, 4H), 7.11 (m, 2H), 7.22 (m, 4H), 7.56 (m, 2H).

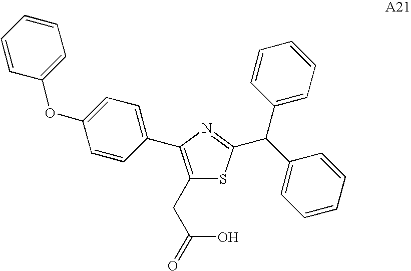

[2-Benzhydryl-4-(4-phenoxy-phenyl)-thiazol-5-yl]-acetic acid. LC/MS (an10p8): Rt 4.04 min, m/z 478 [M+H]; ¹H NMR (CDCl₃): δ 3.88 (s, 2H), 5.87 (s, 1H), 7.07 (m, 4H), 7.14 (m, 1H), 7.25-7.41 (m, 12H), 7.58 (m, 2H).

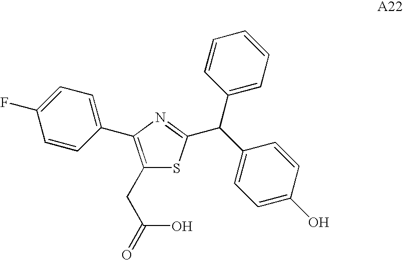

{4-(4-Fluoro-phenyl)-2-[(4-hydroxy-phenyl)-phenyl-methyl]-thiazol-5-yl}-acetic acid. ¹H NMR (CDCl₃): δ 3.77 (s, 2H), 5.82 (s, 1H), 6.70 (m, 2H), 7.02-7.15 (m, 5H), 7.26-7.30 (m, 4H), 7.53 (m, 2H).

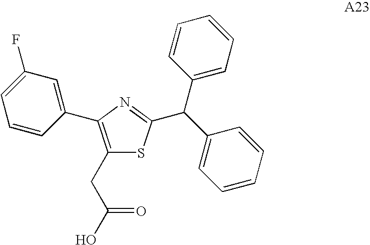

[2-Benzhydryl-4-(3-fluoro-phenyl)-thiazol-5-yl]-acetic acid. LC/MS (an10p8): Rt 2.69 min, m/z 404 [M+H]; ¹H NMR (CDCl₃): δ 3.88 (s, 2H), 5.90 (s, 1H), 7.07 (m, 1H), 7.26-7.41 (m, 13H).

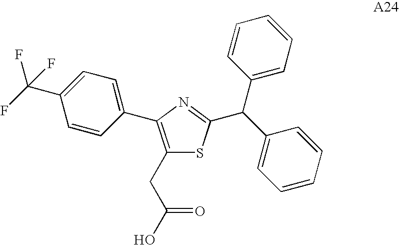

[2-Benzhydryl-4-(4-trifluoromethyl-phenyl)-thiazol-5-yl]-acetic acid. LC/MS (an10p8): Rt 3.09 min, m/z 455 [M+H]; ¹H NMR (CDCl₃): δ 3.91 (s, 2H), 5.91 (s, 1H), 7.34 (m, 10H), 7.72 (m, 4H).

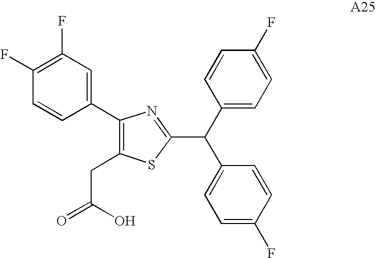

[2-[Bis-(4-fluoro-phenyl)-methyl]-4-(3,4-difluoro-phenyl)-thiazol-5-yl]-acetic acid. LC/MS (an10p8): Rt 3.18 min, m/z 458 [M+H]; ¹H NMR (CDCl₃): δ 3.88 (s, 2H), 5.81 (s, 1H), 7.04 (m, 4H), 7.18-7.36 (m, 6H), 7.46 (m, 1H).

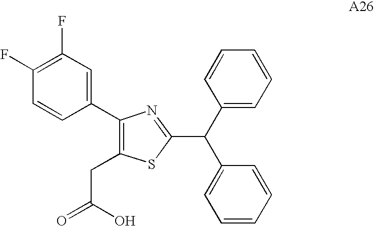

[2-Benzhydryl-4-(3,4-difluoro-phenyl)-thiazol-5-yl]-acetic acid. LC/MS (an10p8): Rt 2.88 min, m/z 422 [M+H]; ¹H NMR (CDCl₃): δ 3.88 (s, 2H), 5.81 (s, 1H), 7.20 (m, 1H), 7.26-7.37 (m, 9H), 7.48 (m, 1H).

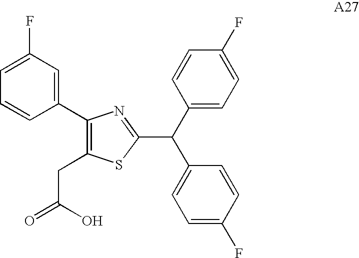

[2-[Bis-(4-fluoro-phenyl)-methyl]-4-(3-fluoro-phenyl)-thiazol-5-yl]-acetic acid. LC/MS (an10p8): Rt 3.08 min, m/z 440 [M+H]; ¹H NMR (CDCl₃): δ 3.92 (s, 2H), 5.82 (s, 1H), 7.04 (m, 4H), 7.24-7.29 (m, 6H) and 7.37 (m, 2H).

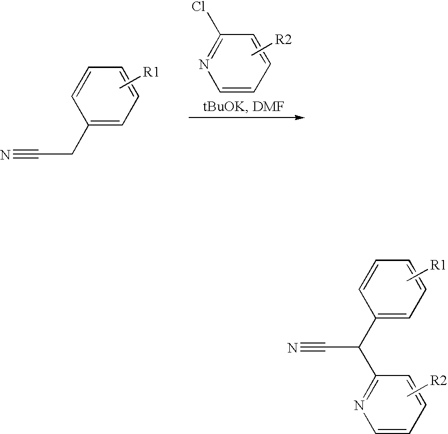

Synthesis of nitrile intermediates—Representative Procedure 7 (RP7): To a cooled (5° C.) solution of Potassium tert-butoxide (2.1 mmol) in dry DMF (0.6 ml) was added a mixture of the benzonitrile (1 mmol) and the 2-Chloro-pyridine (1.1 mmol) in dry DMF (0.4 ml). After O/N stirring at RT, aq. NH₄Cl and EtOAc were added. The organic phase was separated, dried over MgSO₄ and concentrated in vacuo. The residue was purified over silica gel chromatography to give the desired product, which precipitated upon treatment with Et₂O.

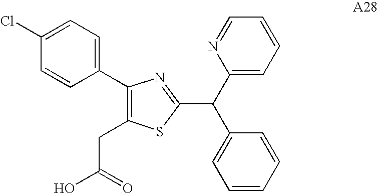

[4-(4-Chloro-phenyl)-2-(phenyl-pyridin-2-yl-methyl)-thiazol-5-yl]-acetic acid. Title compound was prepared from 3-bromo-4-(4-chloro-phenyl)-4-oxo-butyric acid and 2-phenyl-2-pyridin-2-yl-thioacetamide according to RP6 and RP7: LC/MS (an10p8) Rt 3.00 min, m/z 422 [M+H]⁺; ¹H NMR (CDCl₃): δ 3.86 (s, 2H), 6.2 (s, 1H), 7.2-8.0 (m, 12H), 8.7 (d, 1H).

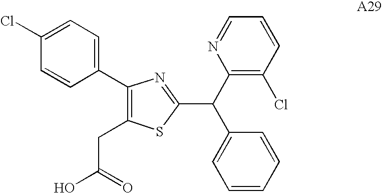

{4-(4-Chloro-phenyl)-2-[(3-chloro-pyridin-2-yl)-phenyl-methyl]-thiazol-5-yl}-acetic acid. Title compound was prepared from 3-bromo-4-(4-chloro-phenyl)-4-oxo-butyric acid and 2-(3-Chloro-pyridin-2-yl)-2-phenyl-thioacetamide according to RP6 and RP7: LC/MS (an10p8) Rt 3.54 min, m/z 454.5 [M+H]⁺; ¹H NMR (CDCl₃): δ 3.83 (s, 2H), 6.66 (s, 1H), 7.2-7.3 (m, 1H), 7.3-7.4 (m, 5H), 7.5 (d, 4H), 7.7 (dd, 1H), 8.6 (dd, 1H).

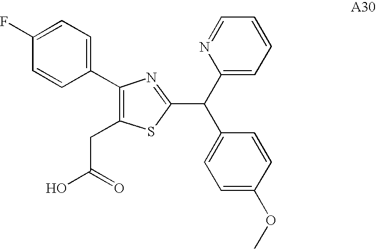

{4-(4-Fluoro-phenyl)-2-[(4-methoxy-phenyl)-pyridin-2-yl-methyl]-thiazol-5-yl}-acetic acid. Title compound was prepared from 3-bromo-4-(4-fluoro-phenyl)-4-oxo-butyric acid and 2-(4-methoxy-phenyl)-2-pyridin-2-yl-thioacetamide according to RP6 and RP7: LC/MS (an10p8) Rt 2.20 min, m/z 434 [M+H]⁺.

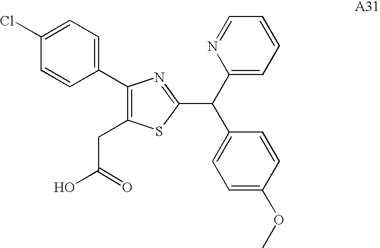

{4-(4-Chloro-phenyl)-2-[(4-methoxy-phenyl)-pyridin-2-yl-methyl]-thiazol-5-yl}-acetic acid. Title compound was prepared from 3-bromo-4-(4-chloro-phenyl)-4-oxo-butyric acid and 2-(4-methoxy-phenyl)-2-pyridin-2-yl-thioacetamide according to RP6 and RP7: LC/MS (an10p8) Rt 2.37 min, m/z 450.5 [M+H]⁺; ¹H NMR (CDCl₃): δ3.75 (s, 3H), 3.78 (s, 2H) 6.0 (s, 1H), 6.9 (d, 2H), 7.2 (t, 1H), 7.3 (m, 4H), 7.4 (d, 1H), 7.5 (d, 2H), 7.6 (dt, 1H), 8.1 (d, 1H).

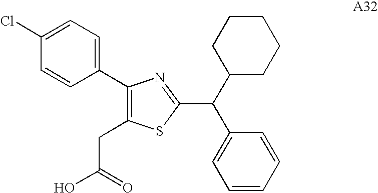

[4-(4-Chloro-phenyl)-2-(cyclohexyl-phenyl-methyl)-thiazol-5-yl]-acetic acid. Title compound was prepared from 3-bromo-4-(4-chloro-phenyl)-4-oxo-butyric acid and 2-cyclohexyl-2-phenyl-thioacetamide according to RP6: LC/MS (an10p8) Rt 3.20 min, m/z 425.4 [M+H]⁺.

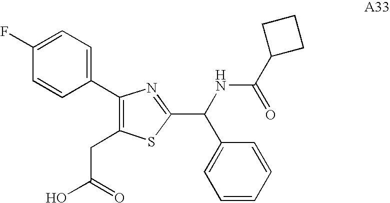

[2-[(Cyclobutanecarbonyl-amino)-phenyl-methyl]-4-(4-fluoro-phenyl)-thiazol-5-yl]-acetic acid. Title compound was prepared from 3-bromo-4-(4-fluoro-phenyl)-4-oxo-butyric acid and 2-cyclobutyl-2-phenyl-thioacetamide according to RP6: LC/MS (an10p8): Rt 2.01 min, m/z 425 [M+H]; ¹H NMR (CDCl₃): δ 1.92 (m, 2H), 2.19 (m, 2H), 3.13 (p, J=8.4 Hz, 1H), 3.81 (s, 2H), 6.49 (d, J=7.5, 1H (NH)), 7.13 (m, 3H), 7.36 (m, 4H) and 7.54 (m, 2H).

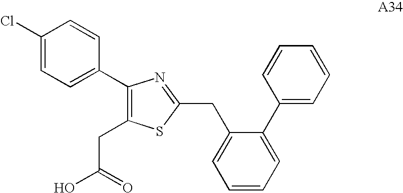

[2-Biphenyl-2-ylmethyl-4-(4-chloro-phenyl)-thiazol-5-yl]-acetic acid. Title compound was prepared from 3-bromo-4-(4-chloro-phenyl)-4-oxo-butyric acid and 2-biphenyl-2-yl-thioacetamide according to RP6: LC/MS (an10p8) Rt 4.94 min, m/z 419.4 [M+H]⁺; ¹H NMR (CDCl₃): δ 3.83 (s, 2H), 4.33 (s, 2H), 7.3-7.5 (m, 13H).

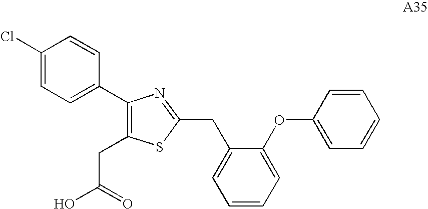

[4-(4-Chloro-phenyl)-2-(2-phenoxy-benzyl)-thiazol-5-yl]-acetic acid. Title compound was prepared from 3-bromo-4-(4-chloro-phenyl)-4-oxo-butyric acid and 2-(2-phenoxy-phenyl)-thioacetamide according to RP6: LC/MS (an10p8) Rt 3.80 min, m/z 435.4 [M+H]⁺; ¹H NMR (CDCl₃): δ 3.82 (s, 2H), 4.39 (s, 2H), 6.9 (d, 1H), 7.0 (d, 2H), 7.1 (m, 2H), 7.2-7.3 (m, 3H), 7.4 (t, 3H), 7.5 (d, 2H).

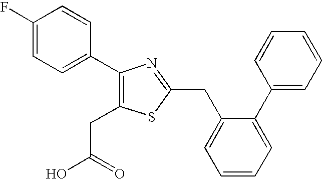

[2-Biphenyl-2-ylmethyl-4-(4-fluoro-phenyl)-thiazol-5-yl]-acetic acid. Title compound was prepared from 3-bromo-4-(4-fluoro-phenyl)-4-oxo-butyric acid and 2-biphenyl-2-yl-thioacetamide according to RP6: LC/MS (an10p8) Rt 2.60 min, m/z 403 [M+H]⁺; ¹H NMR (CDCl₃); δ 3.78 (s, 2H), 4.31 (s, 2H), 7.1 (t, 2H), 7.3-7.5 (m, 11H).

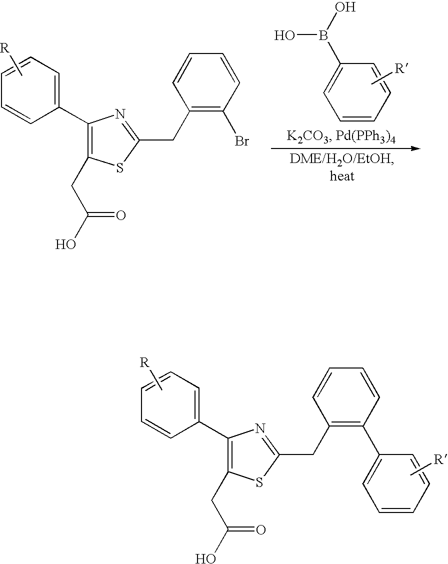

### Representative Procedure 8 (RP8)

A mixture of Pd(PPh₃)₄ (˜50 mg), K₂CO₃ (2 mmol), [2-(2-bromo-benzyl)-thiazol-5-yl]-acetic acid (1 mmol) and the corresponding phenyl boronic acid (1.3 mmol) in DME/H2O/EtOH (7/3/2, 20 mL) was heated at 150° C. for 10 minutes in a microwave. After cooling, water, acetic acid and EtOAc were added. The solid materials were filtered off and the filtrate was concentrated in vacuo. The residue was taken up in hot acetonitrile and solid particles were filtered off. The filtrate was allowed to cool to RT, upon which the desired compound precipitated. In some occasions, the crude material was purified by chromatography.

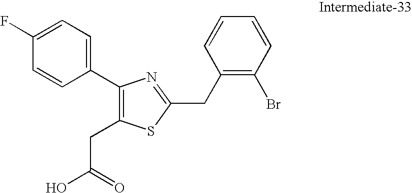

[2-(2-Bromo-benzyl)-4-(4-fluoro-phenyl)-thiazol-5-yl]-acetic acid. Title compound was prepared from 3-bromo-4-(4-fluoro-phenyl)-4-oxo-butyric acid and 2-(2-bromo-phenyl)-thioacetamide according to RP6: LC/MS (an10p8) Rt 2.22 min, m/z 405.4 [M+H]⁺; ¹H NMR (CDCl₃): δ 3.85 (s, 2H), 4.55 (s, 2H), 7.1 (m, 3H), 7.3 (t, 1H), 7.4 (d, 1H), 7.5-7.6 (m, 3H).

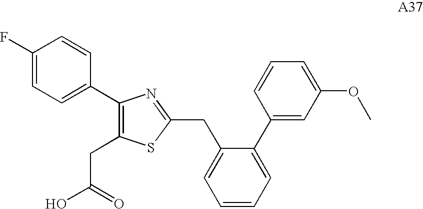

[4-(4-Fluoro-phenyl)-2-(3′-methoxy-biphenyl-2-ylmethyl)-thiazol-5-yl]-acetic acid. Title compound was prepared from [2-(2-bromo-benzyl)-4-(4-fluoro-phenyl)-thiazol-5-yl]-acetic acid and 3-methoxyphenyl boronic acid according to RP8: LC/MS (an10p8) Rt 2.65 min, m/z 433 [M+H]⁺; ¹H NMR (CDCl₃): δ 3.76 (s, 3H), 3.81 (s, 2H), 4.43 (s, 2H), 6.8-6.9 (m, 2H), 7.1 (t, 1H), 7.3-7.5 (m, 7H), 7.7 (m, 2H).

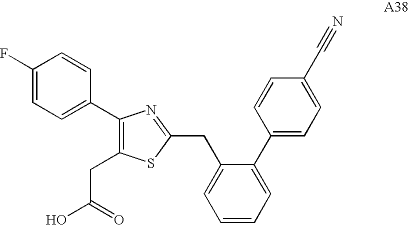

[2-(4′-Cyano-biphenyl-2-ylmethyl)-4-(4-fluoro-phenyl)-thiazol-5-yl]-acetic acid. Title compound was prepared from [2-(2-bromo-benzyl)-4-(4-fluoro-phenyl)-thiazol-5-yl]-acetic acid and 4-cyanophenyl boronic acid according to RP8: LC/MS (an10p8) Rt 2.47 min, m/z 428 [M+H]⁺; ¹H NMR (CDCl₃): δ 3.84 (s, 2H), 4.36 (s, 2H), 7.1 (t, 2H), 7.3-7.5 (m, 8H), 7.7 (d, 2H).

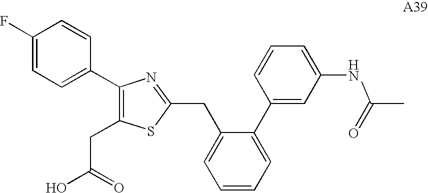

[2-(3′-Acetylamino-biphenyl-2-ylmethyl)-4-(4-fluoro-phenyl)-thiazol-5-yl]-acetic acid. Title compound was prepared from [2-(2-bromo-benzyl)-4-(4-fluoro-phenyl)-thiazol-5-yl]-acetic acid and 3-acetamidobenzene boronic acid according to RP8: LC/MS (an10p8) Rt 2.18 min, m/z 460 [M+H]⁺; ¹H NMR (CDCl₃): δ1.98 (s, 3H), 3.78 (s, 2H), 4.3 (s, 2H), 7.0-7.1 (m, 4H), 7.2-7.4 (m, 2H), 7.4 (m, 4H), 7.6 (m, 2H).

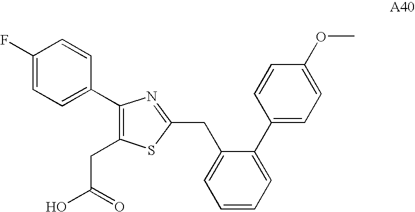

[4-(4-Fluoro-phenyl)-2-(4′-methoxy-biphenyl-2-ylmethyl)-thiazol-5-yl]-acetic acid. Title compound was prepared from [2-(2-bromo-benzyl)-4-(4-fluoro-phenyl)-thiazol-5-yl]-acetic acid and 4-methoxyphenyl boronic acid according to RP8: LC/MS (an10p8) Rt 2.60 min, m/z 433 [M+H]⁺; ¹H NMR (CDCl₃): δ3.82 (s, 2H), 3.84 (s, 3H), 4.37 (s, 2H), 6.9 (d, 2H), 7.1 (t, 2H), 7.2-7.3 (m, 5H), 7.4 (m, 1H), 7.5 (m, 2H).

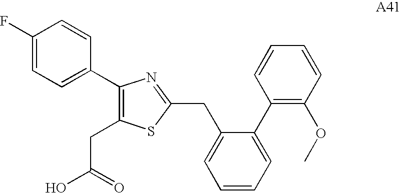

[4-(4-Fluoro-phenyl)-2-(2′-methoxy-biphenyl-2-ylmethyl)-thiazol-5-yl]-acetic acid. Title compound was prepared from [2-(2-bromo-benzyl)-4-(4-fluoro-phenyl)-thiazol-5-yl]-acetic acid and 2-methoxyphenyl boronic acid according to RP8: LC/MS (an10p8) Rt 2.55 min, m/z 433 [M+H]⁺; ¹H NMR (CDCl₃): δ 3.71 (s, 3H), 3.81 (s, 2H), 4.3 (s, 2H), 6.9-7.0 (m, 2H), 7.1 (m, 3H), 7.2-7.4 (m, 4H), 7.4-7.5 (m, 3H).

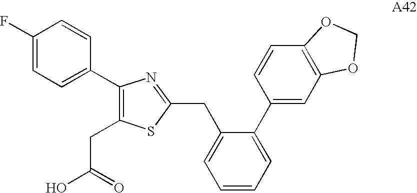

[2-(2-Benzo[1,3]dioxol-5-yl-benzyl)-4-(4-fluoro-phenyl)-thiazol-5-yl]-acetic acid. Title compound was prepared from [2-(2-Bromo-benzyl)-4-(4-fluoro-phenyl)-thiazol-5-yl]-acetic acid and 3,4-methylenedioxobenzene boronic acid according to RP8: LC/MS (an10p8) Rt 3.35 min, m/z 447 [M+H]⁺; ¹H NMR (CDCl₃): δ 3.78 (s, 2H), 4.34 (s, 2H) 5.96 (s, 2H), 6.7 (m, 3H), 7.1 (t, 2H), 7.2-7.3 (m, 3H), 7.3 (d, 1H), 7.5 (t, 2H).

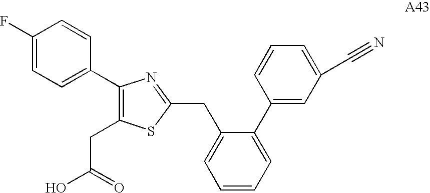

[2-(3′-Cyano-biphenyl-2-ylmethyl)-4-(4-fluoro-phenyl)-thiazol-5-yl]-acetic acid. Title compound was prepared from [2-(2-bromo-benzyl)-4-(4-fluoro-phenyl)-thiazol-5-yl]-acetic acid and 3-cyanophenyl boronic acid according to RP8: LC/MS (an10p8) Rt 3.157 min, m/z 428 [M+H]⁺; ¹H NMR (CDCl₃): δ 3.55 (s, 2H), 3.9 (s, 2H), 6.8 (m, 2H), 7.0 (m, 4H), 7.2-7.4 (m, 6H).

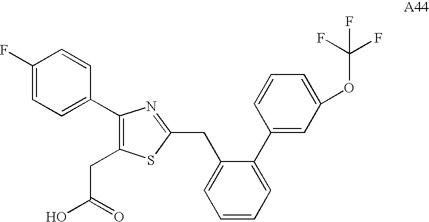

[4-(4-Fluoro-phenyl)-2-(3′-trifluoromethoxy-biphenyl-2-ylmethyl)-thiazol-5-yl]-acetic acid. Title compound was prepared from [2-(2-bromo-benzyl)-4-(4-fluoro-phenyl)-thiazol-5-yl]-acetic acid and 3-trifluoromethoxyphenyl boronic acid according to RP8: LC/MS (an10p8) Rt 3.91 min, m/z 487 [M+H]⁺; ¹H NMR (CDCl₃): δ 3.78 (s, 2H), 4.3 (s, 2H), 7.1 (t, 2H), 7.3 (m, 1H), 7.3 (t, 2H), 7.4-7.5 (m, 7H).

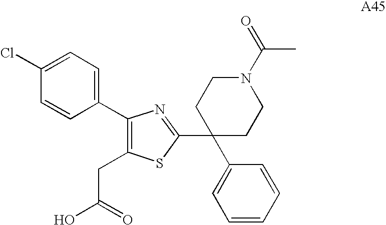

[2-[(1-Acetyl-piperidin-4-yl)-phenyl-methyl]-4-(4-chloro-phenyl)-thiazol-5-yl]-acetic acid. Title compound was prepared from 3-bromo-4-(4-chloro-phenyl)-4-oxo-butyric acid and 1-acetyl-4-phenyl-piperidine-4-carbothioic acid amide 2-(1-acetyl-piperidin-4-yl)-2-phenyl-thioacetamide according to RP6: LC/MS (an10p8) Rt 2.32 min, m/z 454.5 [M+H]⁺.

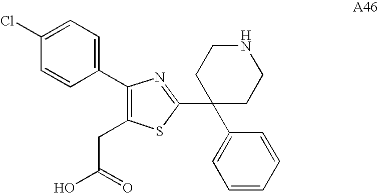

[4-(4-Chloro-phenyl)-2-(4-phenyl-piperidin-4-yl)-thiazol-5-yl]-acetic acid. Title compound, isolated as its HCl salt, was prepared by reacting [2-[(1-acetyl-piperidin-4-yl)-phenyl-methyl]-4-(4-chloro-phenyl)-thiazol-5-yl]-acetic acid with 4N aq. HCl at 95° C. for 18 hours, followed by evaporation of solvent: LC/MS (an10p8) Rt 2.07 min, m/z 412.4 [M+H]⁺.

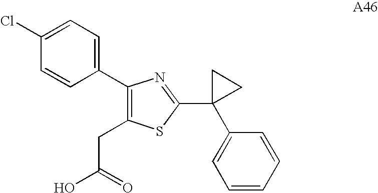

[4-(4-Chloro-phenyl)-2-(1-phenyl-cyclopropyl)-thiazol-5-yl]-acetic acid. Title compound was prepared from 3-bromo-4-(4-chloro-phenyl)-4-oxo-butyric acid and 1-phenyl-cyclopropanecarbothioic acid amide according to RP6: LC/MS (an10p8) Rt 3.57 min, m/z 369.8 [M+H]⁺.

### General Synthetic Route to Imidazole Analogs

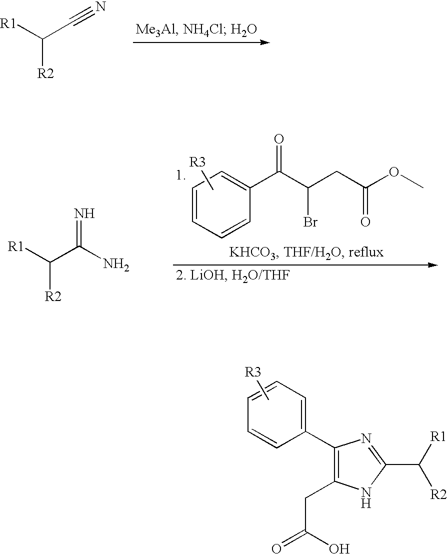

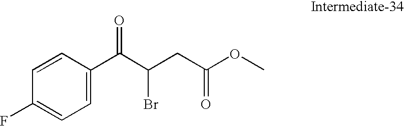

3-Bromo-4-(4-fluorophenyl)-4-oxo-butyric acid methyl ester. 3-Bromo-4-(4-fluorophenyl)-4-oxobutyric acid (15 mmol) was dissolved in methanol (60 ml) and thionyl chloride (17 mmol) was added at 0° C. After stirring at 80° C. for 2 h the solvent was evaporated. The residue was stripped with diethylether. The product was used directly in the next step: ¹H NMR (CDCl₃): δ 3.00 (m, 1H), 3.33 (m, 1H), 3.67 (s, 3H), 5.44 (t, 1H), 7.15 (t, 2H), 8.06 (t, 2H).

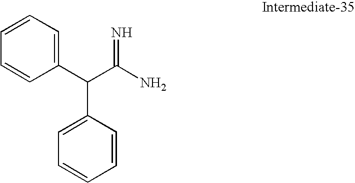

2,2-Diphenylacetamidine. Prepared from diphenylacetonitrile according to R. A. Moss, W. Ma, D. C. Merrer, and S. Xue (Tetrahedron Lett. 1995, 36, 8761-8764): LC/MS (an10p8): Rt 1.57 min, m/z 211 [M+H]⁺.

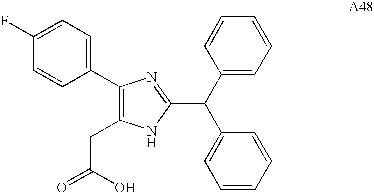

[2-Benzhydryl-5-(4-fluoro-phenyl)-3H-imidazol-4-yl]-acetic acid. A suspension of 2,2-diphenylacetamidine (10 mmol) and potassium carbonate (38 mmol) in THF/H2O (150 mL/50 mL) was heated to reflux. 3-Bromo-4-(4-fluorophenyl)-4-oxo-butyric acid methyl ester (10 mmol) in THF (50 mL) was added slowly. The reaction mixture was refluxed for 16 h, and then concentrated. The residue was dissolved in water (10 mL) and extracted with CH₂Cl₂ (10 mL). The organic phase was dried (MgSO₄) and concentrated to produce [2-benzhydryl-5-(4-fluoro-phenyl)-3H-imidazol-4-yl]-acetic acid methyl ester: LC/MS (an10p8): Rt 3.64 min, m/z 401 [M+H]⁺. The methyl ester in THF was added excess LiOH.H₂O in water. The reaction was stirred at room temperature over night, 3% HCl was added until pH<1, and the mixture was extracted with CH₂Cl₂. The organic phase was dried (MgSO₄) and concentrated to give the product was subject to hydrolysis with LiOH in water/THF: LC/MS (an10n8): Rt 1.43 min, m/z 387 [M−H]⁻.

### Biological Assays

### Materials and Methods

Generation/origin of the cDNA Constructs. The coding sequence of the human CRTH2 receptor (genbank accession no NM_{—}004778) was amplified by PCR from a human hippocampus cDNA library and inserted into the pcDNA3.1(+) expression vector (invitrogen) via 5′ HindIII and 3′ EcoRI. To generate a CRTH2-Renilla luciferase (CRTH2-Rluc) fusion protein, the CRTH2 coding sequence without a STOP codon and Rluc were amplified, fused in frame by PCR and subcloned into the pcDNA3.1(+)Zeo expression vector (invitrogen). Human β-arrestin2 (β-arr2) N-terminally tagged with GFP² (βarr2-GFP²) and Renilla luciferase were purchased from BioSignal Packard Inc, (Montreal, Canada). The sequence identity of the construct was verified by restriction endonuclease digests and sequencing in both directions on an ABI Prism (Applied Biosystems, Foster City, Calif.).

Sequence ID CRTH2 (protein sequence):
MSANATLKPLCPILEQMSRLQSHSNTSIRYIDHAAVLLHGLASLLGLVEN
GVILFVVGCRMRQTVVTTWVLHLALSDLLASASLPFFTYFLAVGHSWELG
TTFCKLHSSIFFLNMFASGFLLSAISLDRCLQVVRPVWAQNHRTVAAAHK
VCLVLWALAVLNTVPYFVFRDTISRLDGRIMCYYNVLLLNPGPDRDATCN
SRQAALAVSKFLLAFLVPLAIIASSHAAVSLRLQHRGRRRPGRFVRLVAA
VVAAFALCWGPYHVFSLLEARAHANPGLRPLVWRGLPFVTSLAFFNSVAN
PVLYVLTCPDMLRKLRRSLRTVLESVLVDDSELGGAGSSRRRRTSSTARS
ASPLALCSRPEEPRGPARLLGWLLGSCAASPQTGPLNRALSSTSS
Sequence ID CRTH2 (nucleotide sequence):
atgtcggc caacgccaca ctgaagccac tctgccccat
cctggagcag atgagccgtc tccagagcca cagcaacacc
agcatccgct acatcgacca cgcggccgtg ctgctgcacg
ggctggcctc gctgctgggc ctggtggaga atggagtcat
cctcttcgtg gtgggctgcc gcatgcgcca gaccgtggtc
accacctggg tgctgcacct ggcgctgtcc gacctgttgg
cctctgcttc cctgcccttc ttcacctact tcttggccgt
gggccactcg tgggagctgg gcaccacctt ctgcaaactg
cactcctcca tcttctttct caacatgttc gccagcggct
tcctgctcag cgccatcagc ctggaccgct gcctgcaggt
ggtgcggccg gtgtgggcgc agaaccaccg caccgtggcc
gcggcgcaca aagtctgcct ggtgctttgg gcactagcgg
tgctcaacac ggtgccctat ttcgtgttcc gggacaccat
ctcgcggctg gacgggcgca ttatgtgcta ctacaatgtg
ctgctcctga acccggggcc tgaccgcgat gccacgtgca
actcgcgcca ggcggccctg gccgtcagca agttcctgct
ggccttcctg gtgccgctgg cgatcatcgc ctcgagccac
gcggccgtga gcctgcggtt gcagcaccgc ggccgccggc
ggccaggccg cttcgtgcgc ctggtggcag ccgtcgtggc
cgccttcgcg ctctgctggg ggccctacca cgtgttcagc
ctgctggagg cgcgggcgca cgcaaacccg gggctgcggc
cgctcgtgtg gcgcgggctg cccttcgtca ccagcctggc
cttcttcaac agcgtggcca acccggtgct ctacgtgctc
acctgccccg acatgctgcg caagctgcgg cgctcgctgc
gcacggtgct ggagagcgtg ctggtggacg acagcgagct
gggtggcgcg ggaagcagcc gccgccgccg cacctcctcc
accgcccgct cggcctcccc tttagctctc tgcagccgcc
cggaggaacc gcggggcccc gcgcgtctcc tcggctggct
gctgggcagc tgcgcagcgt ccccgcagac gggccccctg
aaccgggcgc tgagcagcac ctcgagttag

Cell Culture and Transfection. COS-7 cells were grown in Dulbecco's modified Eagle's medium (DMEM) 1885 supplemented with 10% fetal bovine serum, 100 units/ml penicillin, 1000 μg/ml streptomycin, and kept at 37° C. in a 10% CO₂ atmosphere. HEK293 cells were maintained in Minimum Essential medium (MEM) supplemented with 10% (v/v) heat inactivated fetal calf serum (HIFCS), 2 mM Glutamax™-I, 1% non essential amino acids (NEAA), 1% sodium pyruvate and 10 μg/ml gentamicin. For binding experiments, COS7 cells were transiently transfected with the CRTH2 receptor using a calcium phosphate-DNA coprecipitation method with the addition of chloroquine (as described by Hoist et al., 2001*). To perform the functional Bioluminescence Resonance Energy Transfer (BRET) assays, a HEK293 cell clone stably expressing βarr2-GFP² and CRTH2-Rluc was generated (CRTH2-HEK293 cells).

Binding assay. 24 h after transfection COS-7 cells were seeded into 96 well plates at a density of 30.000 cells/well. Competition binding experiments on whole cells were then performed about 18-24 h later using 0.1 nM [³H]PGD2 (NEN, 172 Ci/mmol) in a binding buffer consisting of HBSS (GIBCO) and 10 mM HEPES. Competing ligands were diluted in DMSO which was kept constant at 1% (v/v) of the final incubation volume. Total and nonspecific binding were determined in the absence and presence of 10 μM PGD2. Binding reactions were routinely conducted for 3 h at 4° C. and terminated by 2 washes (100 μl each) with ice cold binding buffer. Radioactivity was determined by liquid scintillation counting in a TOPCOUNTER (Packard) following over night incubation in Microscint 20. Stable HEK293 cells were seeded at a density of 30.000 cells/well 18-24 h prior to the binding assay which was performed essentially as described for COS7 cells above. Determinations were made in duplicates.

BRET assay. Functional BRET assays were performed on HEK293 cells stably expressing human CRTH2-Rluc and GFP²-β-arr2. Prior to their use in the BRET assay cells were detached and re-suspended in D-PBS with 1000 mg/L L-Glucose at a density of 2×10⁶ cells/mL. DeepBlueC™ was diluted to 50 μM in D-PBS with 1000 mg/L L-Glucose (light sensitive). 100 μL of cell suspension was transferred to wells in a 96-well microplate (white OptiPlate) and placed in the Mithras LB 940 instrument (BERTHOLD TECHNOLOGIES, Bad Wildbad, Germany). 12 μL/well agonist was then injected by injector 1 and 10 μL/well DeepBlueC™ was injected simultaneously by injector 2. Five seconds after the injections the light output from the well was measured sequentially at 400 nm and 515 nm, and the BRET signal (mBRET ratio) was calculated by the ratio of the fluorescence emitted by GFP²-β-arr2 (515 nm) over the light emitted by the receptor-Rluc (400 nm). Antagonists were added before placing the microplates into the Mithras LB 940 and allowed to incubate for 15 minutes prior to the addition of agonist and DeepBlueC™. Compounds were dissolved in DMSO and the final DMSO concentration was kept constant at 1% in the assay.

Human eosinophil shape change assay. Blood was sampled from healthy volunteers according to a protocol approved by the Ethics Committee of the University of Graz and processed as described previously (Bohm et al., 2004). Preparations of polymorphonuclear leukocytes (containing eosinophils and neutrophils) were prepared by dextran sedimentation of citrated whole blood and Histopaque gradients. The resulting cells were washed and resuspended in assay buffer (comprising PBS with Ca²⁺/Mg²⁺ supplemented with 0.1% BSA, 10 mM HEPES and 10 mM glucose, pH 7.4) at 5×10⁶ cells/mL. Cells were incubated with the antagonists or vehicle (PBS or DMSO) for 10 min at 37° C. and then stimulated with various concentration of the agonists (PGD2 or eotaxin) for 4 min at 37° C. To stop the reaction, samples were transferred to ice and fixed with 250 μL of fixative solution. Samples were immediately analyzed on a FACSCalibur flow cytometer (Becton Dickinson) and eosinophils were identified according to their autofluorescence in the FL-1 and FL-2 channels. Shape change responses were quantified as percentage of the maximal response to PGD2 or eotaxin in the absence of an antagonist.

### Materials

Tissue culture media and reagents were purchased from the Gibco invitrogen corporation (Breda, Netherlands). PGD2 was obtained from Cayman and [3H]PGD2 from NEN.

### Data Analysis

Curve analysis was performed with the GraphPadPrism software 3.0 (Graphpad Prism Inc., San Diego, USA) and IC₅₀ values were calculated as a measure of the antagonistic potencies.

### REFERENCES

Hoist B, Hastrup H, Raffetseder U, Martini L, Schwartz T W. Two active molecular phenotypes of the tachykinin NK1 receptor revealed by G-protein fusions and mutagenesis. J Biol Chem. 2001 Jun. 8; 276(23):19793-9. Epub 2001 Feb 22.

### Biological Data:

Compounds were tested in the receptor binding assay and the functional antagonist assay described below, and their IC₅₀ values were assessed. The compounds are grouped in three classes:

### A: IC₅₀ value lower than 0.5 μM

### B: IC₅₀ value between 0.5 μM and 5 μM

### C: IC₅₀ value higher than 5 μM

Tables 1 to 4 give the biological test results for the compounds synthesised above and for some additional compounds acquired from commercial sources.

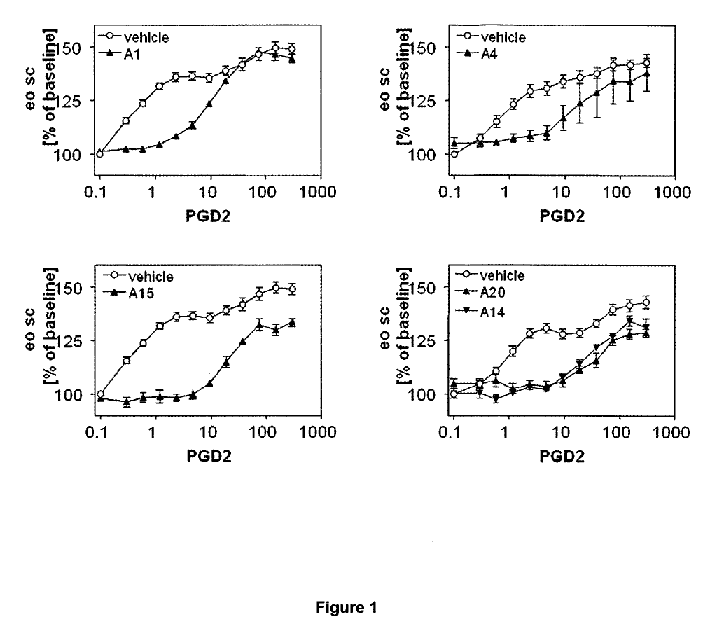

The ability of the compounds above to inhibit prostaglandin D2 induced eosihophil shape change is demonstrated by the examples in FIG. 1

TABLE 1

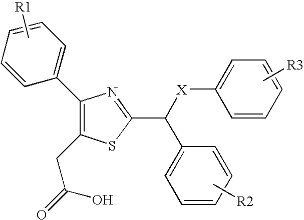

| | | | | | Binding | Antag. |
|---|---|---|---|---|---|---|
| | X | R1 | R2 | R3 | IC₅₀ | IC₅₀ |
| A1 | Bond | 4-Cl | H | H | A | A |
| A4 | Bond | 4-F | H | H | A | A |
| A5 | CH₂ | 4-Cl | H | H | A | B |
| A7 | CH₂ | 4-F | 4-Cl | H | A | A |
| A8 | Bond | 4-Cl | 4-Cl | H | A | A |
| A9 | CH₂ | 4-Cl | 4-Cl | H | A | B |
| A10 | Bond | 4-F | 4-Cl | H | A | A |
| A11 | Bond | H | H | H | B | A |
| A12 | Bond | 4-MeO | H | H | A | B |
| A13 | Bond | 4-Cl | 4-F | H | A | A |
| A14 | Bond | 4-F | 4-F | 4-F | A | A |
| A15 | Bond | 4-F | 4-MeO | H | A | A |
| A16 | Bond | 4-Cl | 4-MeO | H | A | A |
| A17 | Bond | 4-Cl | 3,4-DiF | H | A | C |
| A18 | Bond | 4-F | 3,4-DiF | H | A | A |
| A19 | NHCO | 4-F | H | 4-CF₃ | A | C |
| A20 | Bond | 4-F | 4-MeO | 4-MeO | A | A |
| A21 | Bond | 4-PhO | H | H | A | B |
| A22 | Bond | 4-F | 4-OH | H | A | A |
| A23 | Bond | 3-F | H | H | A | A |
| A24 | Bond | 4-CF₃ | H | H | B | C |
| A25 | Bond | 3,4-DiF | 4-F | 4-F | A | A |
| A26 | Bond | 3,4-DiF | H | Ph | A | A |
| A27 | Bond | 3-F | 4-F | 4-F | A | A |

TABLE 2

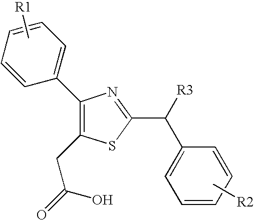

| | | | | Binding | Antag. |
|---|---|---|---|---|---|
| | R1 | R2 | R3 | IC₅₀ | IC₅₀ |
| A28 | 4-Cl | H | 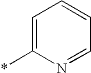 | A | B |
| A29 | 4-Cl | H | 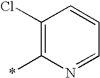 | A | |
| A30 | 4-F | 4-MeO | 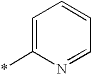 | A | |
| A31 | 4-Cl | 4-MeO | 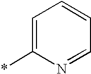 | A | A |
| A32 | 4-Cl | H | 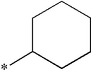 | B | |
| A6 | 4-Cl | H | Et | A | |

TABLE 3

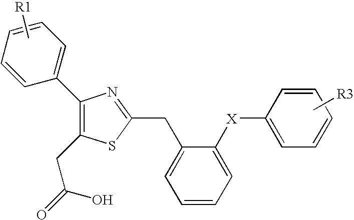

| | | | | Binding | Antag. |
|---|---|---|---|---|---|
| | X | R1 | R2 | IC₅₀ | IC₅₀ |
| A34 | Bond | 4-Cl | H | A | A |
| A35 | O | 4-Cl | H | B | |
| A36 | Bond | 4-F | H | A | A |
| A37 | Bond | 4-F | 3-MeO | A | A |
| A38 | Bond | 4-F | 4-CN | A | A |
| A39 | Bond | 4-F | 3-NHAc | B | |
| A40 | Bond | 4-F | 4-MeO | A | A |
| A41 | Bond | 4-F | 2-MeO | A | |
| A42 | Bond | 4-F | 3,4-OCH₂O | A | A |
| A43 | Bond | 4-F | 3-CN | A | |
| A44 | Bond | 4-F | 3-OCF₃ | A | |

TABLE 4

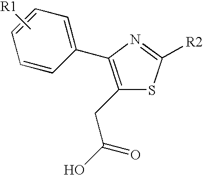

| | | | Binding | Antag. |
|---|---|---|---|---|
| | R1 | R2 | IC₅₀ | IC₅₀ |
| A45 | 4-Cl | 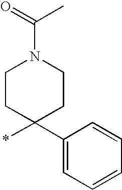 | A | B |
| A46 | 4-Cl | 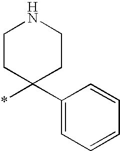 | B | C |

1. A compound of formula (I) or a salt, hydrate or solvate thereof: 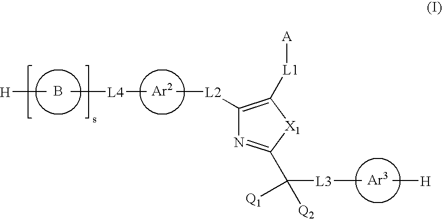
wherein
X₁ is —S—, —O—, —N═N—. —NR₇—, —CR₇═CR₈—, —CR₇═N—, wherein R₇ and R₈ are independently hydrogen or C₁-C₃ alkyl;
A is a carboxyl group —COOH, or a carboxyl bioisostere;
rings Ar² and Ar³ each independently represent a phenyl or 5- or 6-membered monocyclic heteroaryl ring, or a bicyclic ring system consisting of a 5- or 6-membered carbocyclic or heterocyclic ring which is benz-fused or fused to a 5- or 6-membered monocyclic heteroaryl ring, said ring or ring system being optionally substituted;
ring B is as defined for Ar² and Ar³, or an optionally substituted N-pyrrolidinyl, N-piperidinyl or N-azepinyl ring;
s is 0 or 1;
L1 represents a divalent radical of formula -(Alk¹)ₘ- and L2 and L4 each independently represents a divalent radical of formula -(Alk¹)ₘ-(Z)ₙ-(Alk²)ₚ
wherein
m, n and p are independently 0 or 1,
Alk¹ and Alk² are independently optionally substituted straight or branched chain C₁-C₃ alkylene or C₂-C₃ alkenylene radicals which may contain a compatible —O—, —S— or —NR— link wherein R is hydrogen or C₁-C₃ alkyl, and
Z is —O—; —S—; —C(═O)—; —SO₂—; —SO—; —NR—, —NRSO₂—, —C(═O)NR—, —NRCONH—, NRC(═NR)NH—, or ═N—NR— wherein R is hydrogen or C₁-C₃ alkyl; or a divalent 5- or 6-membered monocyclic carbocyclic or heterocyclic radical;
L3 represents a divalent radical of formula -(Alk³)ₘ-(Z)ₙ-(Alk²)ₚ- wherein m, n, p, Alk² and Z are as defined in relation to L2 and L4, and Alk3 is an optionally substituted straight or branched chain C₁-C₂ alkylene or C₁-C₂ alkenylene radical which may contain a compatible —O—, —S— or —NR— link wherein R is hydrogen or C₁-C₃ alkyl;
Q₁ represents hydrogen or (C₁-C₆)alkyl;
Q₂ represents
(i) (C₁-C₆)alkyl, (C₁-C₆)alkoxy, hydroxy, hydroxy(C₁-C₆)alkyl, nitrile (—CN), phenyl, phenoxy, monocyclic heteroaryl or heteroaryloxy with 5 or 6 ring atoms, —CONR^{A}R^{B}, —NR^{B}COR^{A}, —NR^{B}SO₂R^{A} or —NR^{A}CONR^{A}R^{B} wherein R^{A} and R^{B} are independently hydrogen or a (C₁-C₆)alkyl group, or R^{A} and R^{B} are linked to the same N atom to form a cyclic amino ring, and when Q is phenyl, phenoxy or monocyclic heteroaryl or heteroaryloxy with 5 or 6 ring atoms the phenyl or heteroaryl ring is optionally substituted by any of (C₁-C₆)alkyl, (C₁-C₆)alkoxy, hydroxy, hydroxy(C₁-C₆)alkyl, (C₁-C₃)alkylthio, halo, fully or partially fluorinated (C₁-C₃)alkyl, (C₁-C₃)alkoxy or (C₁-C₃)alkylthio, trifluoromethylthio, nitro, nitrile (—CN), —COOR^{A}, —COR^{A}, —OCOR^{A}, —SO₂R^{A}, —CONR^{A}R^{B}, —SO₂NR^{A}R^{B}, NR^{A}R^{B}, —NR^{B}COR^{A}, NR^{B}COOR^{A}, —NR^{B}SO₂R^{A} or —NR^{A}CONR^{A}R^{B} wherein R^{A} and R^{B} are independently hydrogen or a (C₁-C₆)alkyl group, or R^{A} and R^{B} are linked to the same N atom to form a cyclic amino ring, or
(ii) hydrogen, but only when, in L3, Z represents an optionally substituted divalent 5- or 6-membered monocyclic carbocyclic or heterocyclic radical;
or Q₁ and Q₂ taken together with the carbon atom to which they are attached form a C₃-C₆ cycloalkyl ring or a monocyclic non-aromatic heterocyclic ring with 4-6 ring atoms;
and wherein the total length of L2 and L3 does not exceed that of an unbranched saturated chain of 10 carbon atoms.

2. A compound as claimed in claim 1 wherein (i) the length of each of L2, L3 and L4 does not exceed that of an unbranched saturated chain of 5 atoms and (ii) the total length of L2, L3 and L4 does not exceed that of an unbranched saturated chain of 7 atoms, and (iii) none of L1, L2, L3 and L4 includes more than two R substituents different from hydrogen.

3. A compound as claimed in claim 1 wherein L3 is a bond, Q₁ is hydrogen, and Q₂ is phenyl or monocyclic heteroaryl with 5 or 6 ring atoms optionally substituted by any of (C₁-C₆)alkyl, (C₁-C₆)alkoxy, hydroxy, hydroxy(C₁-C₆)alkyl, (C₁-C₃)alkylthio, halo, fully or partially fluorinated (C₁-C₃)alkyl, (C₁-C₃)alkoxy or (C₁-C₃)alkylthio, trifluoromethylthio, nitro, nitrile (—CN), —COOR^{A}, —COR^{A}, —OCOR^{A}, —SO₂R^{A}, —CONR^{A}R^{B}, —SO₂NR^{A}R^{B}, —NR^{A}R^{B}, —NR^{B}COR^{A}, —NR^{B}COOR^{A}, —NR^{B}SO₂OR^{A} or —NR^{A}CONR^{A}R^{B} wherein R^{A} and R^{B} are independently hydrogen or a (C₁-C₆)alkyl group, or R^{A} and R^{B} are linked to the same N atom to form a cyclic amino ring.

4. A compound as claimed in claim 1 wherein L3 is a bond, Q₁ is hydrogen, and Q₂ is phenyl, optionally substituted by any of fluoro, chloro, bromo, (C₁-C₃)alkyl, trifluoromethyl, (C₁-C₃)alkoxy, trifluoromethoxy, trifluoromethylthio, dimethylamino, cyano, (C₁-C₃alkyl)SO₂—, NH₂SO₂—, (C₁-C₃alkyl)NHSO₂—, (C₁-C₃alkyl)₂NSO₂—, —CONR^{A}R^{B}, and —NR^{B}COR^{A}. wherein R^{A} and R^{B} are independently hydrogen or a (C₁-C₆)alkyl group, or R^{A} and R^{B} are linked to the same N atom to form a cyclic amino ring.

5. A compound as claimed in claim 1 wherein L3 is —CH₂—, —O—, —S—, —SO₂—, —NHC(═O)—, —CH═CH—, —NR₁₁—, or —NR₁₁CH₂—, wherein R₁₁ is hydrogen or C₁-C₃ alkyl.

6. A compound as claimed in claim 1 wherein Q₂ is hydrogen and L3 represents a divalent radical of formula -(Alk³)ₘ-(Z)ₙ-(Alk²)ₚ- wherein m is 0, n is 1, and Z is a phenylene radical optionally substituted by one or more of fluoro, chloro, bromo, (C₁-C₃)alkyl, trifluoromethyl, (C₁-C₃)alkoxy, trifluoromethoxy, trifluoromethylthio, dimethylamino, cyano, (C₁-C₃alkyl)SO₂—, NH₂SO₂—, (C₁-C₃alkyl)NHSO₂—, (C₁-C₃alkyl)₂NSO₂—, —CONR^{A}R^{B}, and —NR^{B}COR^{A}. wherein R^{A} and R^{B} are independently hydrogen or a (C₁-C₆)alkyl group, or R^{A} and R^{B} are linked to the same N atom to form a cyclic amino ring.

7. A compound as claimed in claim 6 wherein Z is a 1,2-phenylene radical optionally substituted by one or more of fluoro, chloro, bromo, (C₁-C₃)alkyl, trifluoromethyl, (C₁-C₃)alkoxy, trifluoromethoxy, trifluoromethylthio, dimethylamino, cyano, (C₁-C₃alkyl)SO₂—, NH₂SO₂—, (C₁-C₃alkyl)NHSO₂—, (C₁-C₃alkyl)₂NSO₂—, —CONR^{A}R^{b}, and —NR^{B}COR^{A} wherein R^{A} and R^{B} are independently hydrogen or a (C₁-C₆)alkyl group, or R^{A} and R^{B} are linked to the same N atom to form a cyclic amino ring.

8. A compound as claimed in claim 6 wherein Q₁ is hydrogen.

9. A compound as claimed in claim 1 wherein X₁ is —S—.

10. A compound as claimed in claim 1 wherein A is a carboxyl group —COOH.

11. A compound as claimed in claim 1 wherein A is a carboxyl bioisostere selected from —SO₂NHR and —P(═O)(OH)(OR) wherein R is hydrogen methyl or ethyl, —SO₂OH, —P(═O)(OH)(NH₂), —C(═O)NHCN and groups of formulae:

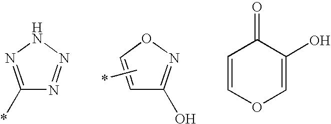

12. A compound as claimed in claim 1 wherein L1 represents a bond, —CR₁₁R₁₂—, *—CH₂CR₁₁R₁₂—, *—CR₁₁R₁₂—, *—SCR₁₁R₁₂—, *—NR₁₁CH₂— or —NR₁₁— wherein R₁₁ and R₁₂ are independently hydrogen or C₁-C₃ alkyl, the bond marked with an asterisk being the one connected to the ring containing X¹.

13. A compound as claimed in claim 1 wherein L1 represents —CH₂— or —CH(CH₃)—.

14. A compound as claimed in claim 1 wherein Ar³ is phenyl, thienyl, naphthyl or 2-, 3- or 4-pyridyl, any of which is optionally substituted.

15. A compound as claimed in claim 14 wherein optional substituents in Ar³ are selected from fluoro, chloro, bromo, (C₁-C₃)alkyl, trifluoromethyl, (C₁-C₃)alkoxy, trifluoromethoxy, trifluoromethylthio, dimethylamino, cyano, (C₁-C₃alkyl)SO₂—, NH₂SO₂—, (C₁-C₃alkyl)NHSO₂—, (C₁-C₃alkyl)₂NSO₂—, —CONR^{A}R^{B}, and NR^{B}COR^{A} wherein R^{A} and R^{B} are independently hydrogen or a (C₁-C₆)alkyl group, or R^{A} and R^{B} are linked to the same N atom to form a cyclic amino ring.

16. A compound as claimed in claim 1 wherein L2 is a bond and Ar² is an optionally substituted phenyl, thienyl, furanyl, pyrrolyl or pyridyl ring.

17. A compound as claimed in claim 16 wherein optional substituents in Ar² are selected from fluoro, chloro, bromo, (C₁-C₃)alkyl, trifluoromethyl, (C₁-C₃)alkoxy, trifluoromethoxy, trifluoromethylthio, dimethylamino, cyano, (C₁-C₃alkyl)SO₂—, NH₂SO₂—, (C₁-C₃alkyl)NHSO₂—, (C₁-C₃alkyl)₂NSO₂—, —CONR^{A}R^{B}, and —NR^{B}COR^{A} wherein R^{A} and R^{B} are independently hydrogen or a (C₁-C₆)alkyl group, or R^{A} and R^{B} are linked to the same N atom to form a cyclic amino ring.

18. A compound as claimed in claim 1 wherein s is 0.

19. A compound of formula (IA), or a salt, hydrate or solvate thereof: 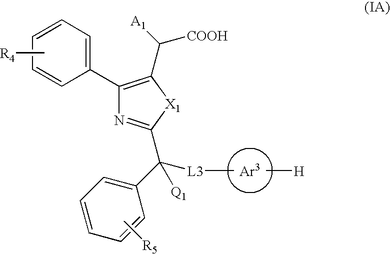
wherein A₁ is hydrogen or methyl, X₁, Q₁, Ar³ and L3 are as defined in claim 1, and R₄ and R₅ independently represent hydrogen or one or more optional substituents.

20. A compound as claimed in claim 19 wherein A₁ is hydrogen.

21. A compound as claimed in claim 19 wherein Q₁ is hydrogen.

22. A compound as claimed in claim 19 wherein X₁ is —S—.

23. A compound as claimed in claim 19 wherein Ar³ is optionally substituted phenyl.

24. A compound as claimed in claim 19 wherein L3 is a bond, —O—, —S—, or —NR— wherein R is hydrogen or C₁-C₃ alkyl.

25. A compound as claimed in claim 19 wherein A₁ is hydrogen, Q₁ is hydrogen, X₁ is —S—, Ar³ is optionally substituted phenyl and L3 is a bond.

26. A compound as claimed in claim 19 wherein optional substituents R₄ and R₅ and optional substituents in Ar³ are independently selected from fluoro, chloro, bromo, (C₁-C₃)alkyl, trifluoromethyl, (C₁-C₃)alkoxy, trifluoromethoxy, trifluoromethylthio, dimethylamino, cyano, (C₁-C₃alkyl)SO₂—, NH₂SO₂—, (C₁-C₃alkyl)NHSO₂—, (C₁-C₃alkyl)₂NSO₂—, —CONR^{A}R^{B}, and NR^{B}COR^{A} wherein R^{A} and R^{B} are independently hydrogen or a (C₁-C₆)alkyl group, or R^{A} and R^{B} are linked to the same N atom to form a cyclic amino ring.

27. A compound of formula (IB), or a salt, hydrate or solvate thereof: 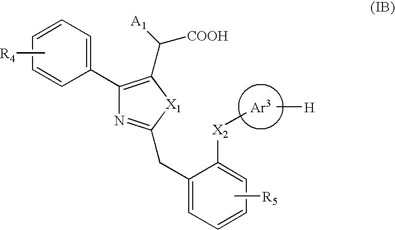
wherein A₁ is hydrogen or methyl, X₂ is a bond, —CH₂—, —O—, —S—, or —NR— wherein R is hydrogen or C₁-C₃ alkyl and X₁ and Ar³ are as defined in claim 1, and R₄ and R₅ independently represent hydrogen or one or more optional substituents.

28. A compound as claimed in claim 27 wherein A₁ is hydrogen.

29. A compound as claimed in claim 27 wherein X₁ is —S—.

30. A compound as claimed in claim 27 wherein Ar³ is optionally substituted phenyl.

31. A compound as claimed in claim 27 wherein X₂ is —CH₂— or a bond.

32. A compound as claimed in claim 27 wherein optional substituents R₄ and R₅ and optional substituents in Ar³ are independently selected from fluoro, chloro, bromo, (C₁-C₃)alkyl, trifluoromethyl, (C₁-C₃)alkoxy, trifluoromethoxy, trifluoromethylthio, dimethylamino, cyano, (C₁-C₃alkyl)SO₂—, NH₂SO₂—(C₁-C₃alkyl)NHSO₂—, (C₁-C₃alkyl)₂NSO₂—, —CONR^{A}R^{B}, and —NR^{B}COR^{A} wherein R^{A} and R^{B} are independently hydrogen or a (C₁-C₆)alkyl group, or R^{A} and R^{B} are linked to the same N atom to form a cyclic amino ring.

33. A compound selected from the group consisting of
[2-benzhydryl-4-(4-chlorophenyl)-thiazol-5-yl]-acetic acid,
[2-benzhydryl-4-(4-fluoro-phenyl)-thiazol-5-yl]-acetic acid,
[2-[1-(4-chloro-phenyl)-2-phenyl-ethyl]-4-(4-fluoro-phenyl)-thiazol-5-yl]-acetic acid,
{4-(4-chloro-phenyl)-2-[(4-chloro-phenyl)-phenyl-methyl]-thiazol-5-yl}-acetic acid,
[2-[(4-chloro-phenyl)-phenyl-methyl]-4-(4-fluoro-phenyl)-thiazol-5-yl]-acetic acid,
[2-[bis-(4-fluoro-phenyl)-methyl]-4-(4-fluoro-phenyl)-thiazol-5-yl]-acetic acid,
{4-(4-fluoro-phenyl)-2-[(4-methoxy-phenyl)-phenyl-methyl]-thiazol-5-yl}-acetic acid,
{4-(4-chloro-phenyl)-2-[(4-methoxy-phenyl)-phenyl-methyl]-thiazol-5-yl}-acetic acid,
[2-[(3,4-difluoro-phenyl)-phenyl-methyl]-4-(4-fluoro-phenyl)-thiazol-5-yl]-acetic acid,
[2-[bis-(4-methoxy-phenyl)-methyl]-4-(4-fluoro-phenyl)-thiazol-5-yl]-acetic acid,
[2-benzhydryl-4-(3-fluoro-phenyl)-thiazol-5-yl]-acetic acid,
[2-[bis-(4-fluoro-phenyl)-methyl]-4-(3,4-difluoro-phenyl)-thiazol-5-yl]-acetic acid,
[2-benzhydryl-4-(3,4-difluoro-phenyl)-thiazol-5-yl]-acetic acid,
[2-[bis-(4-fluoro-phenyl)-methyl]-4-(3-fluoro-phenyl)-thiazol-5-yl]-acetic acid,
and salts hydrates and solvates thereof.

34. A pharmaceutical composition comprising a compound as claimed in claim 1, together with a pharmaceutically acceptable carrier.

35. (canceled)

36. A method of treatment of disease responsive to modulation of CRTH2 receptor activity comprising administering to a subject suffering such disease and effective amount of a compound as claimed in claim 1.

37. A method as claimed in claim 36 wherein the disease is one associated with elevated levels of prostaglandin D2 (PGD2) or one or more active metabolites thereof.

38. A method as claimed in claim 37 wherein the disease is an inflammatory, autoimmune, respiratory or allergy disease.

39. A method as claimed in claim 37 wherein the disease is selected from asthma, rhinitis, allergic airway syndrome, allergic rhinobronchitis, bronchitis, chronic obstructive pulmonary disease (COPD), nasal polyposis, sarcoidosis, farmer's lung, fibroid lung, cystic fibrosis, chronic cough, conjunctivitis, atopic dermatitis, Alzheimer's disease, amyotrophic lateral sclerosis, AIDS dementia complex, Huntington's disease, frontotemporal dementia, Lewy body dementia, vascular dementia, Guillain-Barre syndrome, chronic demyelinating polyradiculoneurophathy, multifocal motor neuropathy, plexopathy, multiple sclerosis, encephalomyelitis, panencephalitis, cerebellar degeneration and encephalomyelitis, CNS trauma, migraine, stroke, rheumatoid arthritis, ankylosing spondylitis, Behget's Disease, bursitis, carpal tunnel syndrome, inflammatory bowel disease, Crohn's disease, ulcerative colitis, dermatomyositis, Ehlers-Danlos Syndrome (EDS), fibromyalgia, myofascial pain, osteoarritis (OA), osteonecrosis, psoriatic arthritis, Reiter's syndrome (reactive arthritis), sarcoidosis, scleroderma, Sjogren's Syndrome, soft tissue disease, Still's Disease, tendinitis, polyarteritis Nodossa, Wegener's Granulomatosis, myositis (polymyositis dermatomyositis), gout, atherosclerosis, lupus erythematosus, systemic lupus erythematosus (SLE), type I diabetes, nephritic syndrome, glomerulonephritis, acute and chronic renal failure, eosinophilia fascitis, hyper IgE syndrome, sepsis, septic shock, ischemic reperfusion injury in the heart, allograft rejection after transplantations, and graft versus host disease.

40. A method as claimed in claim 37 wherein the disease selected from asthma, rhinitis, allergic airway syndrome, and allergic rhinobronchitis.


SEQUENCE LISTING (2 sequences)

SEQ ID NO: 1 - protein, 395 aa, Homo sapiens
MSANATLKPLCPILEQMSRLQSHSNTSIRYIDHAAVLLHGLASLLGLVENGVILFVVGCRMRQTVVTTWVLHLALSDLLASASLPFFTYFLAVGHSWELGTTFCKLHSSIFFLNMFASGFLLSAISLDRCLQVVRPVWAQNHRTVAAAHKVCLVLWALAVLNTVPYFVFRDTISRLDGRIMCYYNVLLLNPGPDRDATCNSRQAALAVSKFLLAFLVPLAIIASSHAAVSLRLQHRGRRRPGRFVRLVAAVVAAFALCWGPYHVFSLLEARAHANPGLRPLVWRGLPFVTSLAFFNSVANPVLYVLTCPDMLRKLRRSLRTVLESVLVDDSELGGAGSSRRRRTSSTARSASPLALCSRPEEPRGPARLLGWLLGSCAASPQTGPLNRALSSTSS

SEQ ID NO: 2 - DNA, 1188 bp, Homo sapiens
atgtcggccaacgccacactgaagccactctgccccatcctggagcagatgagccgtctccagagccacagcaacaccagcatccgctacatcgaccacgcggccgtgctgctgcacgggctggcctcgctgctgggcctggtggagaatggagtcatcctcttcgtggtgggctgccgcatgcgccagaccgtggtcaccacctgggtgctgcacctggcgctgtccgacctgttggcctctgcttccctgcccttcttcacctacttcttggccgtgggccactcgtgggagctgggcaccaccttctgcaaactgcactcctccatcttctttctcaacatgttcgccagcggcttcctgctcagcgccatcagcctggaccgctgcctgcaggtggtgcggccggtgtgggcgcagaaccaccgcaccgtggccgcggcgcacaaagtctgcctggtgctttgggcactagcggtgctcaacacggtgccctatttcgtgttccgggacaccatctcgcggctggacgggcgcattatgtgctactacaatgtgctgctcctgaacccggggcctgaccgcgatgccacgtgcaactcgcgccaggcggccctggccgtcagcaagttcctgctggccttcctggtgccgctggcgatcatcgcctcgagccacgcggccgtgagcctgcggttgcagcaccgcggccgccggcggccaggccgcttcgtgcgcctggtggcagccgtcgtggccgccttcgcgctctgctgggggccctaccacgtgttcagcctgctggaggcgcgggcgcacgcaaacccggggctgcggccgctcgtgtggcgcgggctgcccttcgtcaccagcctggccttcttcaacagcgtggccaacccggtgctctacgtgctcacctgccccgacatgctgcgcaagctgcggcgctcgctgcgcacggtgctggagagcgtgctggtggacgacagcgagctgggtggcgcgggaagcagccgccgccgccgcacctcctccaccgcccgctcggcctcccctttagctctctgcagccgcccggaggaaccgcggggccccgcgcgtctcctcggctggctgctgggcagctgcgcagcgtccccgcagacgggccccctgaaccgggcgctgagcagcacctcgagttag